(19) United States
(12) Patent Application Publication
(10) Pub. No.: US 2025/0124571 A1
(43) Pub. Date: Apr. 17, 2025

(54) MAGNETIC RESONANCE HISTOPATHOLOGY AND NEURAL NETWORKCLASSIFICATION FOR PROSTATE CANCER

(71) Applicant: BIOPROTONICS, INC., Santa barbara, CA (US)

(72) Inventors: Chantal Nguyen, Boulder, CO (US); George Hulsey, Hillsborough, CA (US); Kristin James, Santa Barbara, CA (US); Timothy W. James, Santa Barbara, CA (US); Jean Marie Carlson, Goleta, CA (US)

(21) Appl. No.: 18/914,340

(22) Filed: Oct. 14, 2024

Related U.S. Application Data
(60) Provisional application No. 63/590,428, filed on Oct. 14, 2023.
(60) Provisional application No. 63/675,506, filed on Jul. 25, 2024.

(51) Int. Cl. G06T 7/00; A61B 5/055

(52) U.S. Cl. CPC G06T 7/0012; A61B 5/055; G06T 2207/10088; G06T 2207/20081; G06T 2207/20084; G06T 2207/30081; G06T 2207/30096

(57) ABSTRACT

Diagnosing prostate cancer typically involves invasive measures such as biopsy in which tissue is removed and graded by pathologists. Non-invasive measures like multi-parametric MRI can be used to examine and grade lesions using the PI-RADS scale; however, the achievable image resolution is motion-limited. A new paradigm, magnetic resonance histopathology (MRH), is disclosed which aims to assess tissue texture at sub-millimeter resolution through rapid clinical acquisition procedures. MRH capability of identifying cancerous tissue in the prostate via an in silico analysis is demonstrated, recreating the MRH measurement process on a set of high resolution, pathologist-annotated histology slides. A dataset of spectral intensities at sub-millimeter wavelengths is created and a deep learning model trained to classify spectral data is underlying normal or tumor tissue. A set of spatial frequencies to optimize diagnostic power within the constraint of limited acquisition time is identified. Beyond single-region classification, the disclosed approach and architectures integrate spatial context and local information, the inclusion of which improves model performance and de-noises the inferential results. Applications of the trained models to unlabeled, high-resolution 3D MRI data is demonstrated in addition to algorithms for estimating the physical length scale of lesions identified by the model.

### REFERENCES TO RELATED APPLICATIONS

This application claims the priority benefits of U.S. Provisional application Ser. No. 63/590,428 filed on Oct. 14, 2023 and U.S. Provisional Application Ser. No. 63/675,506 filed on Jul. 25, 2024 both entitled MAGNETIC RESONANCE HISTOPATHOLOGY AND NEURAL NETWORK CLASSIFICATION FOR PROSTATE CANCER and having a common assignee with the present application, the disclosures of which are incorporated herein by reference.

### BACKGROUND

### Field

The disclosed methodology relates to assessment of cancer, describing use for prostate cancer as an example, using Magnetic Resonance Histopathology (MRH) and more particularly to a method for training a neural network for providing a probability vector predicting probability of tumorous tissue with MRH input.

### Related Art

Prostate cancer (PCa) affects millions of people worldwide, with over a million new cases diagnosed every year. The five-year relative survival rate is over 90% when cancer is detected in early stages (comprising about 80% of diagnoses); but can drop to below 40% if the cancer has metastasized outside the prostate. Typically, PCa is diagnosed via biopsy following abnormal results from a digital rectal exam and/or blood test for prostate-specific antigen (PSA). Diagnostic uncertainty, resulting in both over-and undertreatment, remains a major challenge in PCa diagnosis. Systematic transrectal ultrasound-guided biopsy is the standard method of biopsy, but can miss tumors in the anterior regions and carries a risk of infection. More accurate, non-invasive screening methods such as multi-parametric Magnetic Resonance Imaging (mpMRI) can help avoid unnecessary biopsy by better targeting the location; mpMRI achieves better contrast between benign and cancerous tissue than ultrasound, with severe lesions often clearly visible. While mpMRI-guided biopsies are more sensitive to locating clinically significant tumors, they may miss lower-grade cancers; on the other hand, systematic biopsies are more likely to identify indolent tumors that do not require treatment but can miss more severe tumors located in certain regions. A combined mpMRI-targed and systematic biopsy method reduces diagnostic uncertainty, but this procedure requires an increased number of biopsy cores and constitutes an overall increase in time, cost, and infection risk.

Biopsy is a highly invasive procedure, but the resulting histopathology analysis remains the gold standard for diagnosing PCa. H&E-stained histology slides from the biopsy sample are analyzed for Gleason patterns characteristic of cancer and assigned a Gleason score based on the severity of the patterns. In recent years, AI-based methods have supplemented pathologist findings and enabled new scientific discoveries.

### SUMMARY

In a broad example implementation, the disclosure herein provides a method for tuning a neural network for analysis of MRH data wherein a plurality of 2-dimensional regions of interest (ROI) are selected in each of a plurality of histology images or electronic images, each image annotated with normal and tumor regions indication. Each ROI in the plurality of ROIs is then collapsed in a transverse direction associated with a transverse width of the ROI to produce a 1 dimensional signal in an analysis direction associated with an analysis width. The 1 dimensional signal in each ROI is convolved with a set of complex sinusoidal functions to compute a simulated MRH spectrum as a function of wavelength. A simulated spectra is then computed from each ROI in the plurality of ROIs over a plurality of k-values producing a plurality of datasets corresponding to each histology slide in the plurality of histology slides. One or more neighborhoods of each ROI is then selected from the set of 4 nearest neighbors, 8 nearest neighbors 24 nearest neighbors or 16 non-adjacent neighbors. The datasets are then shuffled and split into two portions. The simulated spectra from each ROI in a first one of the portions and the selected neighborhood of that ROI is received in a neural network to produce a two-element probability vector that the input spectrum for that ROI is from normal tissue and the probability that it is from tumor, with these values summing to 1, training the network by comparison of the resulting probability vector with the annotation for that ROI. The simulated spectra from each ROI in a second one of the portions and the selected neighborhood of that ROI is received in the neural network to produce a two-element probability vector that the input spectrum for that ROI is from normal tissue and the probability that it is from tumor, with these values summing to 1 to validate the training of the neural network on the first portion by comparison of the resulting probability vector with the annotation for that ROI. 3D MRH data is then obtained from tissue regions of interest (in vivo or from tissue phantoms) producing actual spectra on a plurality of volumes of interest (VOIs) in a 3D prostrate and the actual spectra from each VOI and a selected neighborhood of that VOI is received in the neural network to produce a two-element probability vector that the spectrum for that VOI is from normal tissue and the probability that it is from tumor, with these values summing to 1.

In a detailed example implementation, the method described provides for MRH measurements by using annotated histology slides to train neural network classifiers. A Pseudo whole-organ digitized H&E-stained histology slide, is created with annotations indicating tumor, Prostatic intraepithelial neoplasia (PIN), and atypical glands. The tumor annotations are converted to an image mask used to label regions of interest as normal or tumor. The whole-organ slide is segmented into the peripheral zone). The peripheral zone is divided into a plurality of regions of interest (ROIs). An example normal ROI is identified, and an ROI located within a tumor is identified. Each ROI in the plurality of ROIs is averaged in one dimension to produce a 1-D position signal, which is then Fourier transformed to produce a simulated MRH spectrum. The spectra from the normal and tumor ROIs are compared, demonstrating notable differences in texture. Because only a limited number of wavelengths can be encoded in a clinical procedure, in an example implementation, a selection of 10 wavelengths, evenly sampled across the spectrum, is identified that enables differentiation between normal and tumor tissue. Intensities at specific wavelengths, extracted from spectra, are used as input. The classifier outputs a vector of probabilities that the corresponding sample is normal or tumor.

The method also employs neighboring-ROI inputs used in order to incorporate the effect of spatial information in the model. In addition to each individual ROI in the plurality of ROIs as selection of one of the following may be incorporated; the ROI with one nearest neighbor on either side is employed, giving a (3,1) input size; the ROI with two neighbors on either side, giving a (5,1) input size; d) the ROI with three neighbors on either side, giving a (7,1) input size; or square-shaped inputs, one of the ROI with one layer of neighbors in all directions, forming a (3,3) sized input; the ROI with two layers of neighbors, forming a (5,5) input size; the ROI with three layers of neighbors, forming a (7,7) input size.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows 2-D histology images used to generate annotated data for in silico analysis;

FIG. 1B shows an example division of histology images into regions of interest (ROI) corresponding to volumes of interest (VOI) in a 3D MRH data acquisition;

FIG. 2 is a schematic of a neural network classifier wherein intensities at specific wavelengths, extracted from spectra, are used as input and the classifier outputs a vector of probabilities that the corresponding sample is normal or tumor;

FIG. 3 is a graph of receiver-operating characteristic (ROC) curves for the neural network output based on intensity at different wavelengths as input;

FIGS. 4A-E are depictions of the neighboring VOIs used in order to incorporate the effect of spatial information in the neural network wherein FIG. 4A shows the VOI (center red rectangle) in isolation; FIG. 4B shows the VOI and its 8 next-nearest neighbors (light blue); FIG. 4C shows the VOI and its 24 nearest neighbors (green); FIG. 4D shows the VOI and its 48 nearest neighbors (yellow); and FIG. 4E the VOI and 16 non-adjacent neighbors (blue);

FIG. 5 is a graph of receiver-operating characteristic (ROC) curves for the neural network using variable amounts of neighboring spectra as described in FIGS. 4A-E demonstrating the best performance is achieved when the spectrum of each VOI is also accompanied by the spectra of 24 or 48 neighbors when input into the network, as demonstrated by the largest area under the curve;

FIG. 6 shows prediction maps for each of the 3 test images, generated with the 0-neighbor, 8-neighbor, and 24-neighbor models placed side-by-side with the ground-truth annotations wherein color scale indicates red for high tumor probability, white for 50% probability of tumor, and blue for low tumor probability;

FIG. 7 shows the receiver-operating characteristic (ROC) curves for the neural network with a VOI and 0 neighbors as input comparing cases in which the VOI is 2 mm×2 mm, 2.5 mm×2 mm, and 3 mm×2 mm in size.

FIG. 8 shows receiver-operating characteristic (ROC) curves for the neural network with a VOI and 24 neighbors as input comparing cases in which the VOI is 2 mm×2 mm, 2.5 mm×2 mm, and 3 mm×2 mm in size;

FIG. 9A shows a 12 mm×7.8 mm×2.7 mm 3D MRI sample, collapsed along z-dimension (2.7 mm);

FIG. 9B shows a prediction map of the 3D MRI sample wherein each VOI is colored with the predicted probability of containing tumor tissue and spectra from each VOI is input without including neighbors;

FIG. 9C is a prediction map with 8 neighbors;

FIG. 9D is a prediction map with 24 neighbors;

FIGS. 10A-10F shows a flow chart wherein MRH measurements are simulated from annotated histology slides and used to train neural network classifiers;

FIG. 11 is representation of a 10 frequency input neural network;

FIG. 12 is a graph of Attribution magnitude of input features averaged over all test sets for the best-performing fully connected neural network with all 39 wavelengths as input features. The longer and shorter wavelengths tend to have a larger effect on network output;

FIG. 13A shows an example ground truth anannotated histology slide with a large carcinoma present (red, tumor; blue, normal);

FIG. 13B shows example model inference results compared to ground truth (red, tumor; blue, normal) ground truth label map for the peripheral zone;

FIG. 13C shows example model inference results compared to ground truth (red, tumor; blue, normal) single-region inference heatmap using a trained neural network;

FIG. 13D shows example model inference results compared to ground truth (red, tumor; blue, normal) inference heatmap from a model which incorporates data from neighboring regions, denoising the model predictions;

FIGS. 14A-H are a graphical depictions of the neighboring-ROI inputs used in order to incorporate the effect of spatial information in the model.

FIG. 15 shows inference results with an increasing number of neighbors in rod configuration, compared with the ground truth labels. Color represents the output predicted probability that a sample contains tumor tissue, with blue (0) indicating a 100% confident prediction that the sample is normal, white (0.5) indicating no confidence that the sample is either normal or tumor, and red (1) indicating a 100% confident prediction that the sample is tumor. Gray represents regions with no data

FIG. 16 shows inference results with the stacked RF model at the F1-optimal classification threshold t; blue is normal, red is abnormal, and gray indicates no data. As the number of neighbors is increased from 2 to 6, the predictions become less noisy and the model becomes more confident. However, the sensitivity to smaller abnormal areas appears to decrease. The ‘no data’ gray region grows as the input size increases, where large inputs cannot be accommodated near the edges of the organ.

FIG. 17 is a graphical comparison of different models for incorporating neighbor information. The cross-validated test AUC-ROC is plotted versus the number of neighboring ROIs included in the input. The CNN performance is divided by evaluation on predicting all 2n +1 labels of the input rod (red) versus predicting only the central ROI (green), which is the sole objective of the stacked RF models. At large input sizes greater than approximately 1 cm in organ, the performance begins to degrade

FIG. 18 shows an isolated tumor sub-array and an inference heatmap generated by the stacked RF model with n=2; this corresponds to an input of 5 ROIs with the goal of predicting the central label;

FIG. 19 is a flowchart for the size estimation algorithm;

FIG. 20 shows tumor size density function obtained from inference results using the stacked RF model and the size estimation algorithm. The 95% confidence interval is shaded in red. The ground truth tumor widths of the sub-array are plotted as a histogram in blue;

FIG. 21 shows in a first panel: 12 mm×15 mm×2 mm 3D MRI sample, collapsed along z-dimension. Second-seventh panels: Prediction maps of the 3D MRI sample, output by models trained on input sizes containing 0 up to 5 neighbors. Each VOI is colored with the predicted probability of containing tumor tissue.

FIG. 22 is a graphical representation of receiver-operating characteristic (ROC) curves for the neural network using intensity at different wavelengths as input. The network trained on 10 wavelengths, evenly spaced between 0.05 and 0.95 mm, is identified as demonstrating the best performance with the lowest chance of overfitting;

FIG. 23 is a graphical representation of receiver-operating characteristic (ROC) curves for the neural network using variable amounts of neighboring spectra aligned in a rod configuration;

FIG. 24 is a graphical representation of distribution of tumor sizes (in connected ROIs) over the entire PZ-segmented dataset. A threshold of 500 connected ROIs (corresponding to a linear scale of ˜7 mm; shown in dashed, black) is chosen to separate the classes ‘small’ and ‘large’;

FIG. 25 is a graphical representation of AUC-ROC for the stacked RF models on the full dataset (black) and the dataset split by tumor size (large tumors in red, small tumors in blue). Performance on large tumors is good while the inclusion of many neighbors decreases performance on small tumors. The AUC metric peaks around n≈16;

FIG. 26 shows the effect of modulating the classification threshold t on the inference maps. 4 thresholds are shown, obtained by perturbing around the F1-optimal threshold t*=0.3. On the left, the tumor sub-array is outlined on the ground truth label map. The inference is done with the stacked RF model with 6 neighboring VOIs;

FIG. 27 shows inference results with an increasing number of neighbors in square configuration;

FIG. 28A presents graphical representation of precision and recall of the stacked RF model versus the number of neighbors n included for a total input size of 2n+1 VOIs for the full dataset; and

FIG. 28B presents the dataset split by tumor sizes small/large, with small tumors defined as comprising less than 500 connected VOIs.

### DETAILED DESCRIPTION

Typical diagnostic procedures for prostate cancer involve invasive measures such as biopsy in which tissue is removed and graded by pathologists. Non-invasive measures such as multi-parametric MRI can be used to examine and grade prostates using the PI-RADS scale, however the image resolution achievable is motion-limited. It is therefore desirable to use MRH, to assess tissue texture at sub-millimeter resolution through rapid clinical acquisition procedures with training of a deep neural network classifier using an in silico analysis employing simulated MRH measurements for assessment of the actual MRH data.

### 1.0 Basic Method

Disclosed herein is a method called magnetic resonance histopathology (MRH) which noninvasively probes tissue texture at a microscopic resolution surpassing that of traditional image-based magnetic resonance imaging (MRI). Pulse sequencing and operational methods for Magnetic Resonance MicroTexture (MRμT) scanning which provide the basis for MRH are disclosed in U.S. Pat. Nos. 10,061,003, 10215827, 10955503 and pending application Ser. No. 17/577,098, all having a common assignee with the present application, the disclosures of which are incorporated herein by reference. Obtaining in vivo images with MRI is highly sensitive to both voluntary and involuntary patient motion such as breathing and bowel peristalsis. The current PI-RADS resolution standard for T2 weighted prostate imaging is a pixel size of ≥0.4 mm (frequency) and ≥0.7 mm (phase) (see Turkbey, Baris, et al., “Prostate Imaging Reporting and Data System Version 2.1: 2019 Update of Prostate Imaging Reporting and Data System Version 2”, European Urology, 76(3): 340-351, 2019 (Turkbey) and Barrett, T., et al. “PI-RADS version 2: what you need to know. Clin Radiol, 70(11): 1165-1176 November 2015) (Barrett)), while glandular structure can vary at a scale of 0.1 mm or less. However, MRH can achieve about one order of magnitude higher resolution (0.05 mm) than traditional MRI, by trading off image acquisition for spectral acquisition that aims to measure texture.

Rather than imaging the organ in two dimensions, MRH uses a one-dimensional signal containing spatial frequency information of the underlying tissue. In one example implementation, MRH is immune to patient motion by measuring a single spatial frequency (k-value) per excitation or a series of k-encodes by modulating the encode for each refocused echo in a multi echo sequence; encoded spins move with the tissue, ensuring accurately localized measurement provided that the excited region remains within the receiver. Repeated excitations are also used to measure multiple k-values (see for example U.S. Pat. No. 10,215,827). While patient motion will occur during this time, spatial coherence is not required, since the goal of MRH is to capture texture rather than a full image. In a second example implementation, MRH may use selective excitation of a first slice within the specimen of interest and a second slice selective refocusing sequence to define a rod followed by application of a gradient along an analysis direction sweeping through a small range of k-values with the receiver bandwidth set narrowly to delineate the length of the volume of interest (VOI) and post processing at various bandwidths to define additional VOIs in the rod along the analysis direction (see for example 10955503).

The measurements using MRH can be thought of as independent but together they provide a picture of the spatial variation in the underlying tissue. Because the tissue is assumed to have a quantifiable texture, that is, a somewhat regularly repeating structure (in prostate, the glands interspersed within stroma) it will have an identifiable spatial frequency signature (spectrum) that corresponds to the salient patterns present in the tissue. Since the time required for data acquisition is directly related to the number of k-values measured, the determination of a small set of informative k-values is a primary goal. Disclosed herein are calibration techniques to find optimal choices of wavelengths which maximize diagnostic capability while minimizing clinical acquisition time.

MRI is a widespread technology, and MRH procedures can be conducted on any MRI scanner. For example, an MRH procedure can be added to an mpMRI scanning, which, in tandem, can produce preliminary diagnostic results and enhance targeting of biopsy locations to minimize unnecessary invasive procedures.

For an example of pulse sequence and operational methods for MRμT as disclosed in pending application Ser. No. 17/577,098 and validation of the method on phantoms and on an ex vivo sample see Sonn, Geoffrey A. et al., “MR method for measuring microscopic histologic soft tissue textures”, Magnetic Resonance in Medicine, 86(1): 308-319, 2021. eprint: https://onlinelibrary.wiley.com/doi/pdf/10.1002/mrm.28731 (Sonn)). Sonn demonstrated stark differences in signal from normal tissue compared to tumor A 114-slice dataset is used and shows systematically that MRμT spectra from normal tissue and tumor can be differentiated using deep learning methods, enabling non-invasive diagnosis and tumor localization.

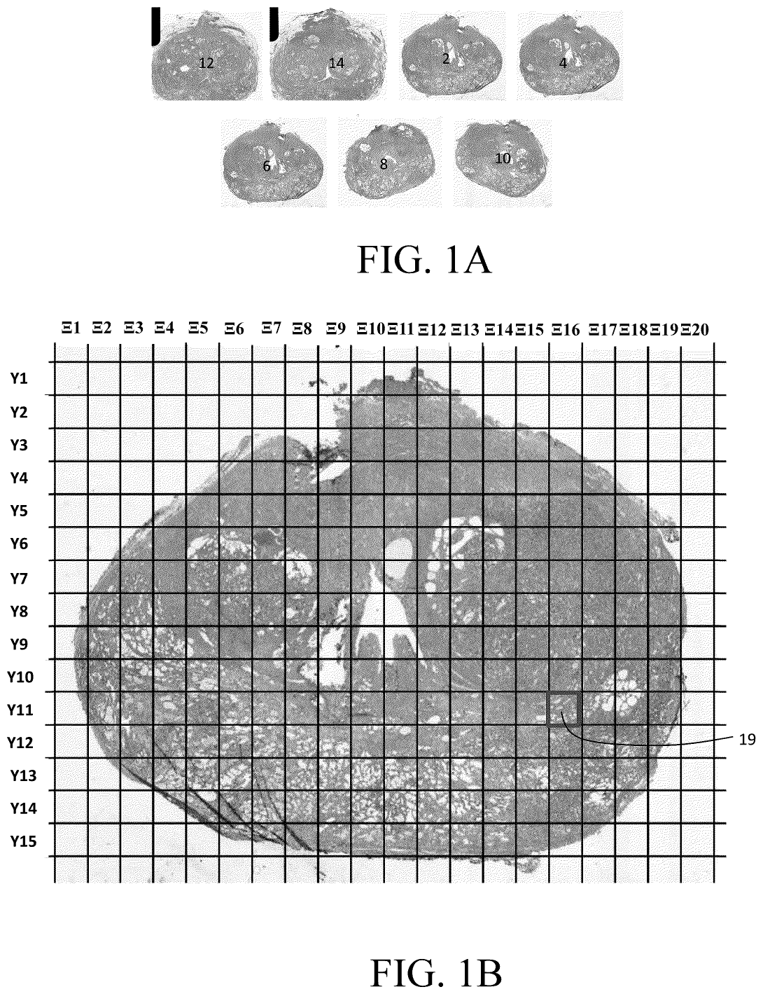

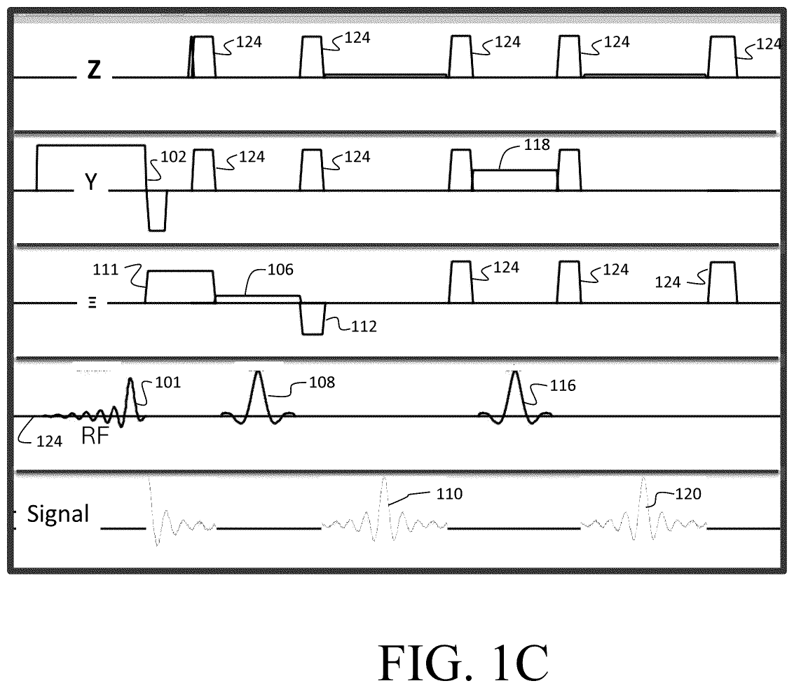

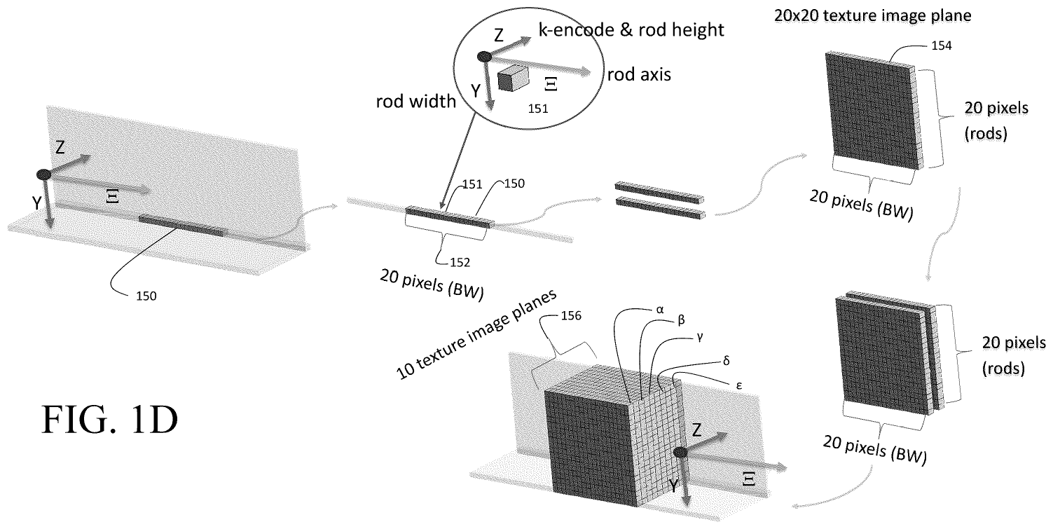

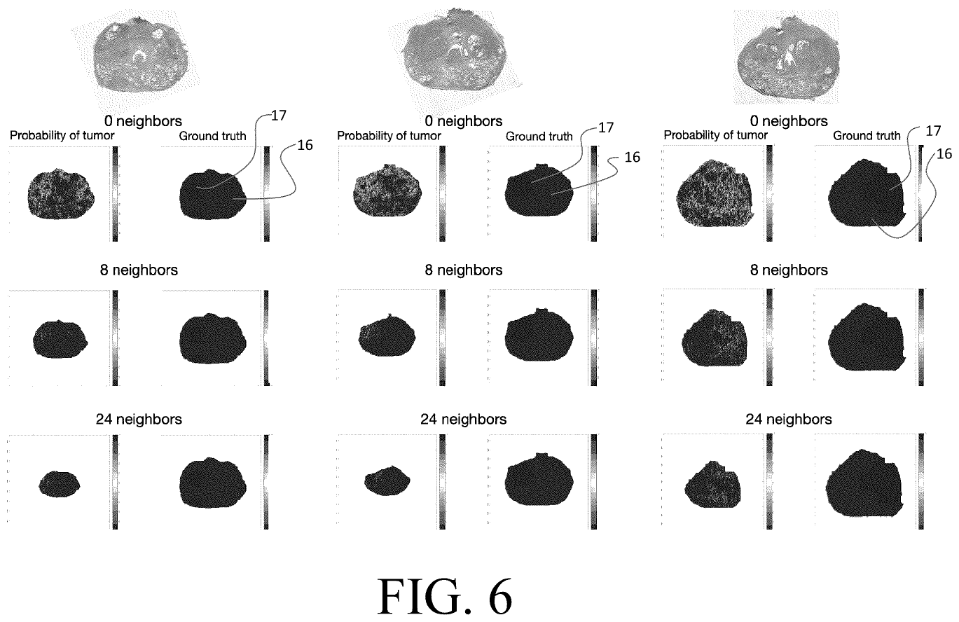

MRH measurements can be simulated from a “ground truth” which may be high-resolution 2-dimensional (2D) annotated histology images, three-dimensional (3D) models created from multiple stacked 2D annotated histology images, or annotated images slices from 3D MRI image data. In a first exemplary implementation described herein, 7 whole-organ histology slides: 5 slides 2, 4, 6, 8 and 10 imaged at 8 μm resolution, and 2 slides 12 and 14 at 16 μm as shown in FIG. 1A. Each image has been annotated by a pathologist with normal regions 16 and tumor regions 17 indicated as will be shown in FIG. 6. After training a model in a neural network on these 2D images, as described subsequently, the model is used to classify a 3D dataset. This 3D data set consists of MRI images of a portion of a prostate as an example organ, imaged at 60 μm×60 μm×90 μm resolution.

### Simulating MRH Data

2-dimensional regions of interest (ROIs) 18 are selected as shown in FIG. 1B from high resolution images of a plurality of histology slides shown in FIG. 1A. “Signal” associated with the image on each slide is determined by pixel color spectral intensity or conversion to grayscale with associated pixel signal. Each ROI is then collapsed to a 1 dimensional (1D) signal by summing signal across one of the two dimensions for the 2D images or, in the case of 3D data discussed below, collapsing along the two orthogonal dimensions, for each increment along an analysis direction, from which a simulated MRH spectrum is computed by convolving the 1D signal with a complex sinusoidal function to compute the simulated MRH spectrum as a function of wavelength. The dimension along which the ROI is collapsed is called the “transverse dimension” 20, and the remaining uncollapsed dimension is called the “analysis dimension” 21. The width of the ROI along the transverse dimension is referred to as the “transverse width”, and the width along the analysis dimension is referred to as the “analysis width”. The transverse and analysis widths are parameters that are optimized for in the in-silico analysis procedure, described below.

An actual MRH pulse sequence (timing diagram) is shown in FIG. 1C. As shown in trace 100, RF pulse 101 is a 90° slice selective RF excitation of a first slab (rod width) with application of a gradient pulse 102 in a first magnetic field gradient defined as the Y axis. A negative gradient pulse 104, rephases the excitation within the defined thickness of the slice or slab. A gradient pulse 106 imposed on the E axis with a 180° RF refocus pulse 108 to define a slab orthogonal to the first slab and generate first spin echo (SE) 110. Preliminary gradient pulse 111 and negative encoding gradient pulse 112 establish a desired k-encode in the analysis direction orthogonal to the second slab (k-encode & rod height). A read gradient 114 is imposed in the Z axis simultaneous to the SE 110. An RF pulse 116 with gradient pulse 118 provides a 180° slice selective refocus to generate multiple echoes 120 (only first one shown) with associated read gradient 122. In addition to the identified gradient pulses there are crusher gradients 124 around all slice selective 180° refocusing pulses.

The resulting structure of the data obtained is shown in FIG. 1D. A selectively excited rod 150 with axis along the E axis and k-encode along the Z axis is defined by the 90° slice selective excitation of first slab to define rod width and 180° slice selective refocus to define rod height and generation of the k-encode along the Z axis by applying the gradient pulse. Imposing a read gradient along the E axis during recording of the spin echo generates a frequency gradient long the rod and provides a means to assign signals to individual pixels 151 representing VOIs along the rod (a 20 pixel bandwidth 152 is shown). The process is then repeated interleaving so that adjacent and coplanar rod acquisitions are separated in time to define texture image planes 154 with 20×20 voxels (pixels) in the Y-E plane and multiple texture image planes parallel to the Y-E plane in the Z direction (10 adjacent texture image planes 156 are shown as an example). The voxel matrix and number of image planes may vary.

The ROI selection in simulated MRH mimics the MRH data gathering procedure: first, “rods” of a fixed width (two dimensional columns with an imposed width in the third dimension) are selected in ground truth images as a simulation of VOIs in actual MRH data and may be considered a simulated VOI. The terms VOI and ROI are therefore used interchangeably herein. The width of these rods correspond to the analysis width. The rods are then divided crosswise into ROIs. The ROIs are collapsed along the transverse dimension and then convolved with a complex sinusoidal function to compute the simulated MRH spectrum as a function of wavelength.

### Neural Network Classification

A neural network is trained on spectra extracted from the 2-D histology slides from ROIs labeled as normal tissue or tumor corresponding to the annotations by the pathologist, examples of which will be described subsequently. In the example implementation, each ROI has an analysis length of 2 mm and a transverse width of 2 mm. To finely sample each image and generate an extensive training data set, ROIs are extracted with a sliding window with a step of 20 pixels (approximately 0.16 mm) over each entire image, and only ROIs that are fully contained within the organ slice are used. Hence, the ROIs will be overlapping in space but correspond to distinct spectra. A total of 208,201 spectra are acquired across a set of 7 images of FIG. 1A for the example implementation described herein.

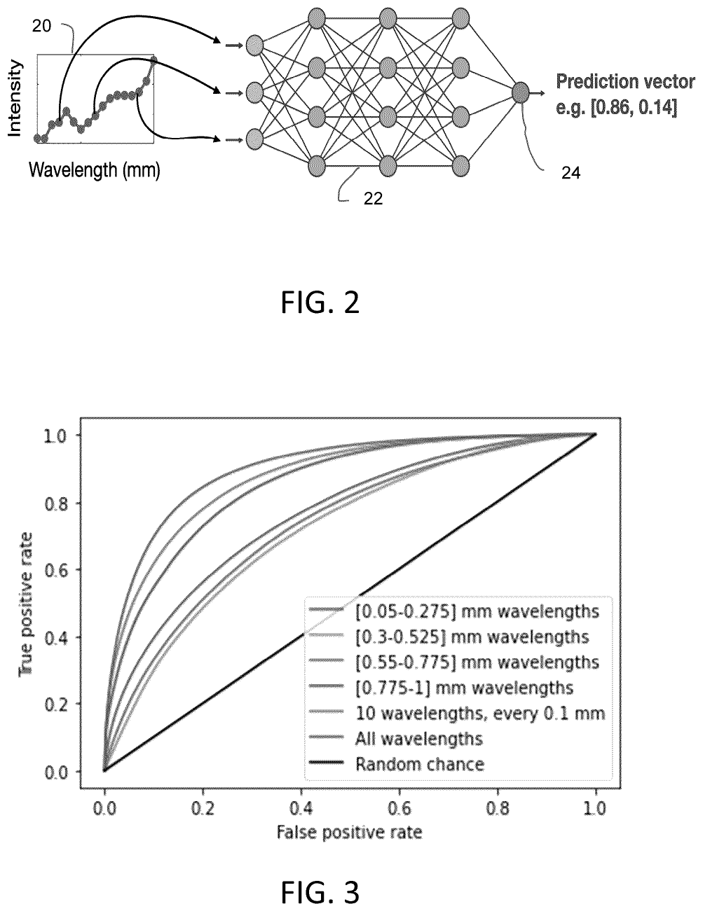

The data for each ROI, one example shown as spectrum 20, is fitted to a 3-layer artificial neural network 22, schematically shown in FIG. 2, with three selected frequency inputs from the spectrum as an example, in order to perform binary classification of spectra as normal or tumor. As will be described subsequently, the number of frequency inputs (k-values) selected for input to the model may vary (see for example FIG. 11 displaying a 10 input representation). In the example implementations the first hidden layer is a 128-node fully-connected layer with ReLU activation; the second is a 64-node fully-connected layer with ReLU activation; the third is a 64-node fully-connected layer with ReLU activation; followed by an output layer with softmax activation for binary classification. An Adam optimizer is used with a learning rate of 0.001. The output of the network is a two-element probability vector 24 containing the probability that the input spectrum is from normal tissue and the probability that it is from tumor, with these values summing to 1.

### Feature Selection

In the in silico analysis, spectra can be obtained from each ROI sampled at a higher number of wavelengths (k-values) than would be typically achieved in a clinical k-encoding procedure. While simulated spectra are computed at about 40 wavelengths, from 0.05 mm to 1 mm in increments of 0.025 mm, a clinical MRH procedure may involve approximately 10 k-encodes, to limit the length of the scan and acquire more samples from a wider region of tissue in the same amount of time. Hence, a feature selection routine is performed to determine which wavelengths are most informative in the binary classification. The performance of neural network classifiers is compared with each classifier trained on 5 sets of different inputs, each containing 10 features: the spectral intensity at a) short wavelengths (between 0.05 and 0.275 mm, in increments of 0.025 mm); b) short-medium wavelengths (between 0.3 and 0.525 mm, in increments of 0.025 mm); c) medium-long wavelengths (between 0.55 and 0.775 mm, in increments of 0.025 mm); d) long wavelengths (between 0.775 and 1 mm, in increments of 0.025 mm); and e) wavelengths spanning the entire range (between 0.05 and 0.95 mm, in increments of 0.01 mm). These results are also compared against a classifier trained on all 40 wavelengths, which would be infeasible to achieve in clinical practice, but demonstrates a potential upper limit on performance.

For each case, a repeated 2-fold cross-validation of the dataset is performed, in which the data is shuffled and split in two portions of equal size. The network is trained and validated on one half of the data and tested on the other. Next, the network is trained on the half that was previously used as test data and tested on the other half. The data is then reshuffled, and this procedure repeated for a total of 10 iterations.

Since the training data possesses a large class imbalance, with many more samples labeled as normal than tumor, unequal weights are applied to each sample to correct this imbalance. ROIs containing tumor samples are assigned a higher class weight than normal samples, defined as the inverse of the prevalence of tumor samples in the training set.

The receiver-operating-characteristic (ROC) curves for each case are compared to determine the performance of the network for each choice of input features as shown in FIG. 3. The network trained on 10 wavelengths, evenly spaced between 0.05 and 0.95 mm, has been identified as demonstrating the best performance with the lowest chance of overfitting. The network performs the worst when only short wavelengths or short-medium wavelengths are used, as characterized by the lower areas under the ROC curves. The network performs much better with long wavelengths, and slightly better with 10 wavelengths sampled across the entire span of wavelengths. Only a small improvement is achieved when all 40 wavelengths are used. Hence, there are likely diminishing returns when more wavelengths are used as input to the network, allowing identification of a regime that satisfies the data-time tradeoff, i.e. a “sweet spot” in which a small number of k-encodes can be performed to achieve high diagnostic power within a short clinical procedure.

### Effect of Spatial Information

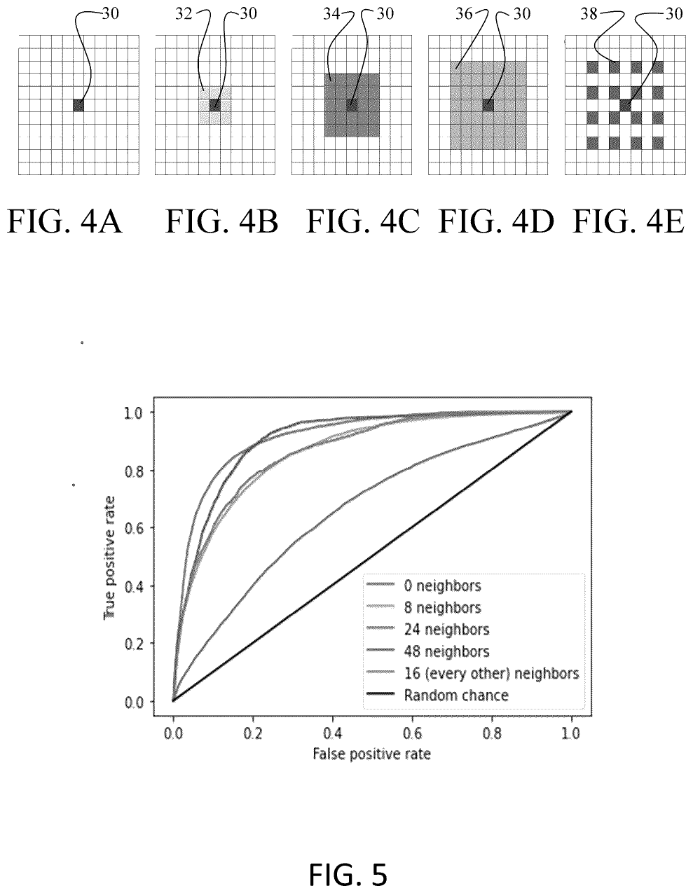

Since regions of normal or cancerous tissue are contiguous and may be associated with characteristic length scales, information from neighboring VOIs as simulated by the spectra of neighboring ROIs of each ROI of the in silico simulation derived from the histology samples as described above can be considered as additional input in the dataset for each ROI into the neural network. The following combinations of inputs as shown in FIGS. 4A-E can be compared: the intensities from 10 wavelengths between 0.05 and 0.95 mm (as described in the previous section) from a) each (2 mm×2 mm) ROI 30 as an independent sample; or with a selected plurality of neighbors. FIG. 4B shows each ROI 30 and its 8 next-nearest neighbors 32, a 6 mm×6 mm region; FIG. 4C shows each ROI 30 and its 24 nearest neighbors 34, a 10 mm×10 mm region; FIG. 4D shows each ROI 30 and its 48 nearest neighbors 36, a 14 mm×14 mm region; and FIG. 4E shows each ROI 30 and 16 non-adjacent neighbors 38, which also spans a 14 mm×14 mm region.

The neural network is trained on the data obtained from a first plurality of images, 4 images in the example shown and the network is tested on data from a second plurality of images, the remaining 3 images in the example shown as seen in FIG. 1A. The same 4(3) images are used for training (testing) for each of the cases listed above. The images 2, 4, 12 and 14 used for training are shown in the top row of FIG. 1A, and images 6, 8 and 10 used for testing are in the bottom row. The performance of the network is evaluated for each case by comparing the ROC curves (FIG. 5). The network performs poorly when the data from each VOI is used as independent inputs; the performance improves greatly when the next 8 nearest neighbors are also included. Using 16 non-adjacent neighbors results in very similar performance.

A further improvement is achieved using 24 neighbors. Similar performance is also achieved using 48 neighbors. A higher true positive rate can be achieved using 24 neighbors when the threshold for false positives is more strict (i.e. more sensitive at a stricter threshold), since the curve rises more rapidly than that of the 48-neighbor model. However, the 24-neighbor curve plateaus at a slightly lower value than the 48-neighbor model. Ultimately, the 24-neighbor model has a slightly higher area under the curve than the 48-neighbor model (0.917 compared to 0.905). It is possible that the larger size of the region spanned by 48 or 16 non-adjacent neighbors contains too much variation in the underlying tissue structure and can wash out rather than amplify the pertinent information contained in the central VOI. In short, 24 neighbors, spanning a 10 mm×10 mm square, results in the best classification performance.

To visualize the output probability of the network, and how this is affected by incorporating spatial information, The output probability is illustrated as heatmaps for each of the test images as shown in FIG. 6 (left columns), side-by-side with the ground-truth pathologist annotations of tumor 17 (right columns) and normal 16. Each pixel corresponds to the output probability that the underlying VOI is tumor (red) or normal (blue), where intermediate (white) values indicate a lack of confidence. The predictions become more confident (fewer white or pale pixels) and also more accurate as the number of neighbors increases.

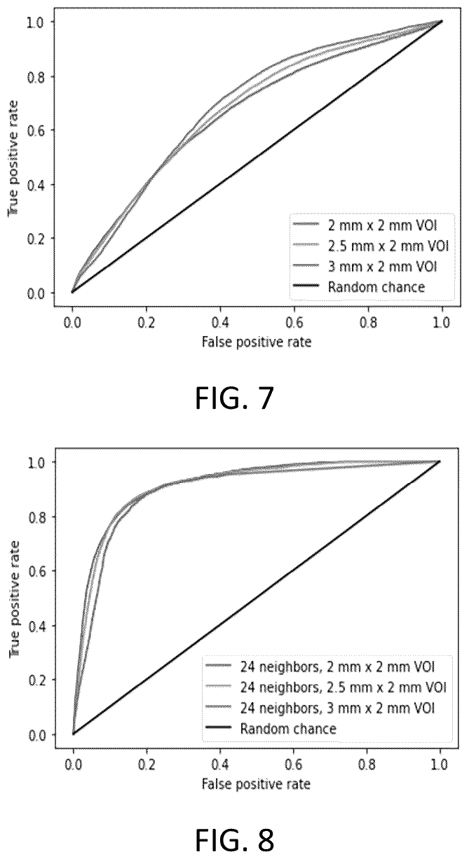

The performance of the underlying VOIs of different sizes are also compared as shown in FIG. 7. The analysis width of each VOI remains fixed to 2 mm, but datasets are also simulated in which the transverse width is 2.5 mm and 3 mm. The results are similar, but the VOI with a transverse width of 3 mm performs slightly better than the other sizes (here each VOI is used in isolation, with no neighbors).

When the 24-neighbor model from the three different VOI sizes is compared (note then that the overall area used as input will vary depending on the VOI size), performance is nearly identical across VOI sizes as seen in FIG. 8.

Application of the method may also be used in 3D where a plurality of sequential histology sample slices α, β, γ, δ, and ε corresponding to planes in the Z axis as shown in FIG. 1D and defining a central “VOI” based on a specific ROI Ξ16, Y11 (designated element 19) shown in the FIG. 1B for a selected sample γ in the middle of the slices and neighbors of the same matrix location in pre and post samples e.g. the VOI of interest is ROI (γ, Ξ16, Y11) and the neighbors are (β, Ξ16, Y11) and (8, 16, Y11) for a 2 neighbor rod and (α, Ξ16, Y11) and (β, 16, Y11); and (δ, Ξ16, Y11) and (ε, Ξ16, Y11) for a 4 neighbor rod.

### Optimal Parameters

In the present example, the optimal parameters for clinical MRH data acquisition that result in best classification performance are as follows:
Approximately 10 wavelengths sampled evenly between [0.05, 1] mm.
Spatial information using 24 neighbors covering a region of up to approximately 10×10 mm.
2 mm analysis width, and 2-3 mm transverse width.

### Inference of 3D Data

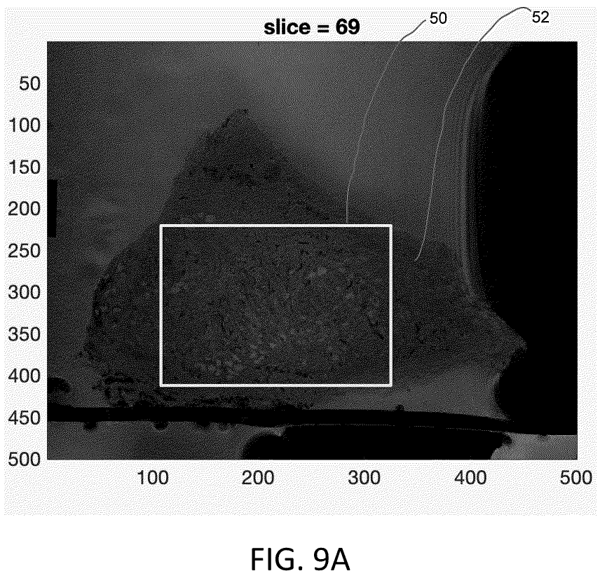

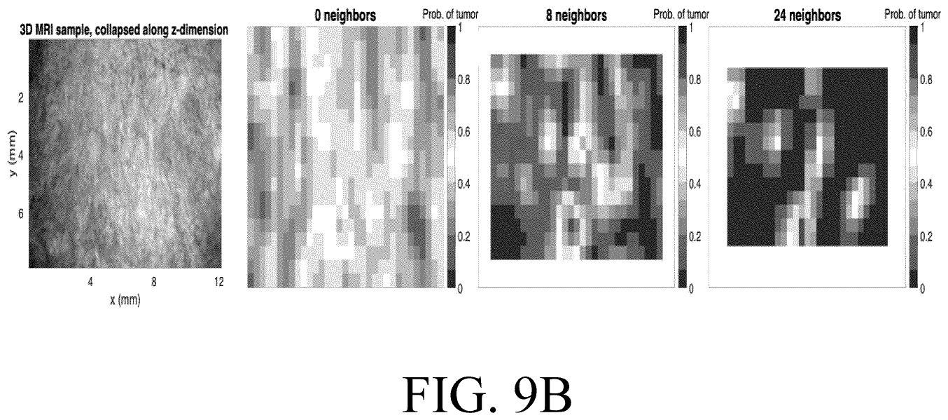

MRH simulation from high resolution 3D MRI images of tissue specimens having a contiguous region 50 of 12 mm×7.8 mm×2.7 mm in the center of an actual organ 52 seen in FIG. 9A are employed to create 3D MRH data. This region is further divided into 3D VOIs that are 2 mm×2 mm×2.7 mm in size (corresponding to ROIs defined in histology specimen images described above). The VOIs are collapsed along the last two dimensions, leaving a 2 mm-long 1-D signal from which a spectrum at wavelengths is generated between 0.15 and 0.95 mm, in increments of 0.1 mm. This matches the wavelengths (k-values) used to train the neural network with histology specimen images described above, with the exception of 0.05 mm (which would not be achievable since the resolution of the 3D MRH data is 0.06 mm along the analysis dimension). The neural network is retrained on these 9 wavelengths extracted from the spectra generated across all seven 2D histology images. The network is then tested on the spectra of the 3D MRI images. Because the 3D MRH data is not annotated in the example shown, ground-truth labeling is not present; however, illustrated in FIG. 9B is a prediction map of the likelihood that each VOI contains tumor tissue. Generation of the prediction map is repeated for cases in which 8 neighboring VOIs are included as input (third panel), and 24 neighboring VOIs (far right panel). (Note that due to the limited size of the 3D tissue region, neighboring VOIs that overlapped were selected in order to generate sufficient data to form a prediction map. The size of the 8-neighbor region is 2.6 mm×2.6 mm, and the size of the 24 neighbor region is 3.2 mm×3.2 mm.) The underlying pattern of the prediction map stays similar with the increase of neighbors, but the prediction probability values sharpen. When 0 neighbors are used, the probabilities are relatively weak values close to 0.5, but the predictions become more confident as the number of neighbors increase, growing closer to either 0 or 1. A predicted tumor region near the bottom-center of the sample remains consistent but becomes more pronounced as the number of neighbors increase.

In an example of using MRI image data as the ground truth images for training the neural network, a plurality of MRI image slices derived from an MRI dataset as described above are annotated by a pathologist with indications of tumor or normal tissue to create a ground truth labeled dataset. The 1D data derived from the plurality of images slices in the 3D MRI data is then employed as input to train the neural network in a manner comparable to that described with regard to the plurality of histology images using cross-validation of the dataset by shuffling and splitting into portions to train, validate and test as described with respect to the histology slide ground truth data above. Training of the neural network can be performed using annotated 2D or 3D annotations of MRI acquired image data, e.g., a radiologist's interpretation or PIRADs report, a pathologist's interpretation of subsequent optical histology of removed tissue, or the determinations from analytical models incorporating conventional MRI image data (e.g., DWI, T1, T2, STIR, etc.)

A neural network trained on any of the annotated images types as described above may then be employed to analyze MRH data obtained in vivo with the previously described MRH pulse format with extracted data corresponding to planes in the Z axis with selected ROIs and neighbors.

### Detailed Experimental Example

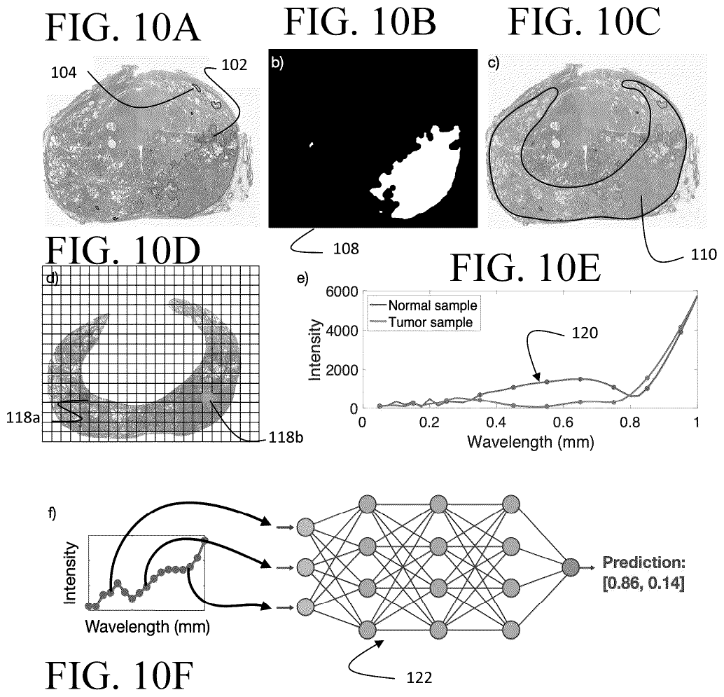

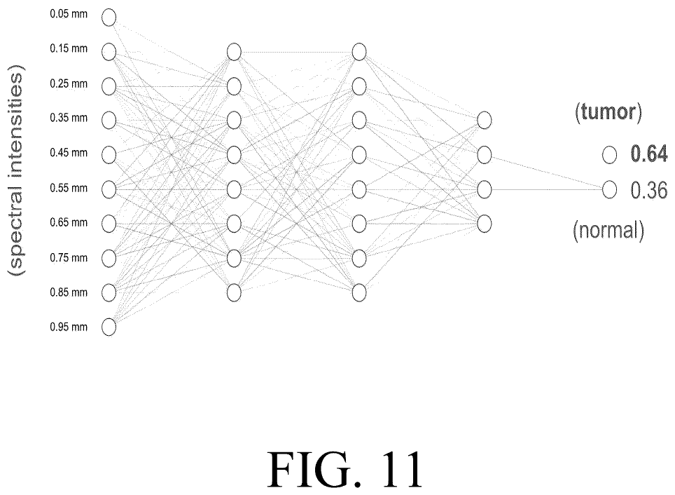

Simulated MRH measurements are disclosed herein which comprise spectral intensities in frequency space taken at selected k-values. Since MRH achieves higher resolution than MRI and is closer to the resolution achievable by histological imaging that can only be done ex vivo, MRH measurements are simulated on histology slides from radical prostatectomies. K-values most informative in classifying tumors are identified using deep learning and develop model architectures for classification of regions into abnormal or normal classes. An overview of the data generation process is shown in FIG. 10A-E wherein step a) provides a pseudo whole-organ digitized H&E-stained histology slide 100 as shown in FIG. 10A, with annotations 102 indicating tumor (red), 104 PIN (blue), and atypical glands 106 (green). In step b) tumor annotations are converted to an image mask 108 seen in FIG. 10B used to label regions of interest as normal or tumor. In step c) the whole-organ slide is segmented into the peripheral zone 110 (outlined and shaded region) as shown in FIG. 10C. In step d) the peripheral zone 110 is divided into 2 mm×2 mm regions of interest (ROIs) 118 as seen in FIG. 10D. An example normal ROI 118a is highlighted in blue, and an ROI 118b located within a tumor is highlighted in orange. In step e) each ROI is averaged in one dimension to produce a 1-D position signal, which is then Fourier transformed to produce a simulated MRH spectrum 120 as shown in FIG. 10E. The spectra from the normal and tumor ROIs 118a, 118b highlighted in FIG. 10D are compared, demonstrating notable differences in texture. Because only a limited number of wavelengths can be encoded in a clinical procedure, a selection of 10 wavelengths, evenly sampled across the spectrum, is identified that enables differentiation between normal and tumor tissue. In step f) the spectral data 120 is provided to the neural network classifier 122 as shown in FIG. 10F (again with three selected frequency inputs from the spectrum as an example. A representation of a 10 frequency input neural network is shown in FIG. 11). Intensities at specific wavelengths, extracted from spectra, are used as input. The classifier outputs a vector 124 of probabilities that the corresponding sample is normal or tumor.

As disclosed herein, the best-performing models reach over 85% classification accuracy. By integrating data from neighboring regions, as described previously, it is shown that the inference results are improved and the predictions are consolidated. Local information can be integrated in a number of ways and different architectures are suggested for doing so. The good performance of these methods for tumor identification on an in silico dataset is shown and confirmation of methods by which the length scale of tumors can be estimated from the simulated MRH measurements is demonstrated. Further, a case study example of applying the disclosed neural network models, trained on simulated MRH data, to an unseen 3D prostate dataset is disclosed.

### Summary of Results

Deep learning models are trained on MRH measurements simulated from “ground truth” histology images, obtained from the dataset described in Singanamalli et al (see Singanamalli, Asha, et al., “Identifying in vivo DCE MRI markers associated with microvessel architecture and Gleason grades of prostate cancer”, Journal of Magnetic Resonance Imaging, 43 (1): 149-158, 2016 (Singanamalli)). The images are annotated by a pathologist with regions labeled as carcinoma, prostatic intraepithelial neoplasia (PIN), or atypical; Only carcinoma annotations are used to label data for training. Furthermore, the dataset is restricted to consist only of the peripheral zone (PZ) of each prostate slice, due to the prevalence of dense stroma outside the PZ that is easily confused with carcinoma. The dense stroma is usually localized to regions that can be excluded during clinical MRH acquisition.

As described previously, the first step of simulating MRH spectral data is dividing each image slice into 2D regions of interest (ROIs), with size comparable to the regions that would be excited within a MRI scanner (FIG. 10D step d). Then, each ROI is collapsed along one dimension to produce a 1D signal from which a measurement of texture is generated. To conduct a clinical MRH measurement, after a volume of tissue is excited, a specific k-value, or spatial frequency/wavelength, is encoded. The signal read from the MR scanner signifies a measure of the extent to which the tissue in the excited region has a texture matching the encoded k-value. With repeated k-encodes, the procedure is extended to obtain a spectrum consisting of intensities over a range of spatial frequencies. To simulate this spectrum, a discrete Fourier transform is applied to the 1D collapsed ROI at specific k-values (or wavelengths) to generate a frequency-space spectrum representative of clinical measurements.

Each spectrum is then labeled according to the tumor annotations (FIG. 10B step b). A threshold is imposed such that if at least 50% of the area of the corresponding ROI lies within a region labeled as carcinoma, then the spectrum is given a positive label (1); otherwise, the spectrum is given a negative label (0) corresponding to the class weights for training data previously described. This threshold can be varied to train models that are more or less sensitive to changes in spectra induced by the presence of tumor; however, setting a threshold too low could increase the false positive rate, while setting a threshold too high could increase the false negative rate. From the resulting dataset, 515,547 individual spectra are achieved, of which 80,475 are labeled as positive (15.6%). This dataset is then used to train deep neural network models to classify spectra as normal or tumor (FIG. 10E step e). A feature selection procedure is first performed to determine the wavelengths, or k-values, that are most informative at differentiating between normal tissue and tumor.

### Feature Selection

Because encoding more k-values increases the length of the MRH scan, and can introduce motion sensitivity, choosing an optimal number of k-values is necessary for determining clinical parameters as well as for training predictive models on simulated data. The number of k-values should be large enough to span a range of textures, enabling differentiation between healthy and cancerous tissue, but also as small as possible in order to minimize scan time. In general, no more than 10 wavelengths is considered to be ideal. Exactly which wavelengths to encode is a critical question for maximizing diagnostic power. Hence, a feature selection experiment is performed in order to determine which wavelengths input to the model result in the best classification performance.

A comparison is disclosed of neural network classifiers each trained on 5 sets of features: the spectral intensity at a) short wavelengths (between 0.05 and 0.275 mm, in increments of 0.025 mm); b) medium-short wavelengths (between 0.3 and 0.525 mm, in increments of 0.025 mm); c) medium-long wavelengths (between 0.55 and 0.775 mm, in increments of 0.025 mm); d) long wavelengths (between 0.775 and 1 mm, in increments of 0.025 mm); and e) wavelengths spanning the entire range (between 0.05 and 0.95 mm, in increments of 0.01 mm). These results are also compared against a classifier trained on 39 wavelengths, between 0.05 and 1 mm in increments of 0.025 mm, which would be infeasible to achieve in clinical practice, but demonstrates a potential upper limit on performance.

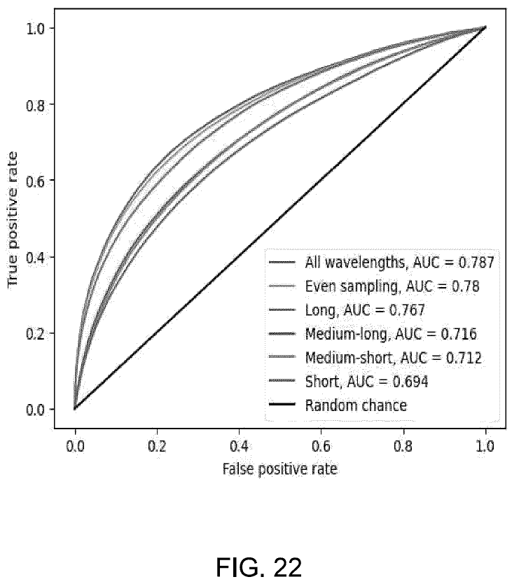

A joint parameter-hyperparameter sweep is performed to determine both the best-performing network architecture for each set of wavelengths, and the best-performing configuration (architecture & features) overall. A comparison is made of the receiver-operating-characteristic (ROC) curves, and the corresponding area under the curve (AUC-ROC), for each case to determine the performance of the network for each choice of input features (FIG. 22). It is observed that the short, medium-short, or medium-long wavelengths result in the worst performance of the model, as quantified by the AUC-ROC. The network performs much better with long wavelengths and slightly better still with 10 wavelengths evenly sampled across the entire span of measurements. The best performance is achieved when all 39 wavelengths are used, but this is only a very small improvement compared to using the 10 evenly-sampled wavelengths. Hence, there are likely diminishing returns when more wavelengths are used as input to the network, allowing us to identify a regime that satisfies the data-time tradeoff, i.e. a “sweet spot” in which a small number of k-encodes can be performed to achieve high diagnostic power within a short clinical procedure. From a deep learning perspective, restricting the input features also combats model overfitting. The best-performing architecture from the model trained on 10 evenly-sampled wavelengths is used as a baseline fully-connected model from which the analysis is expanded to incorporate a wider spatial context, as described in the following subsection.

It is possible that using fewer than 10 wavelengths can achieve a similar level of performance while further reducing measurement time. We address this via a comprehensive parameter sweep of models trained on all possible combinations of 6 and 8 wavelengths from the set of 10 evenly sampled wavelengths identified in the feature selection procedure. The results indicate that even using 6 wavelengths does not dramatically reduce performance. The best performing subset of 6 wavelengths achieves an AUC-ROC of 0.773, while the best performing subset of 8 wavelengths achieves an AUC-ROC of 0.776. In comparison, the best performing subset of 10 wavelengths achieves an AUC-ROC of 0.780. A summary of the best-performing subsets is tabulated in Tables S1 and S2.

TABLE S1
Top 5 best-performing subsets of 6 wavelengths
and corresponding AUC-ROC.

| Wavelengths (mm) | AUC-ROC |
|---|---|
| {0.25, 0.55, 0.65, 0.75, 0.85, 0.95} | 0.773 |
| {0.15, 0.55, 0.65, 0.75, 0.85, 0.95} | 0.771 |
| {0.35, 0.55, 0.65, 0.75, 0.85, 0.95} | 0.771 |
| 0.45, 0.55, 0.65, 0.75, 0.85, 0.95} | 0.770 |
| {0.25, 0.45, 0.65, 0.75, 0.85, 0.95} | 0.770 |

### Attribution Analysis

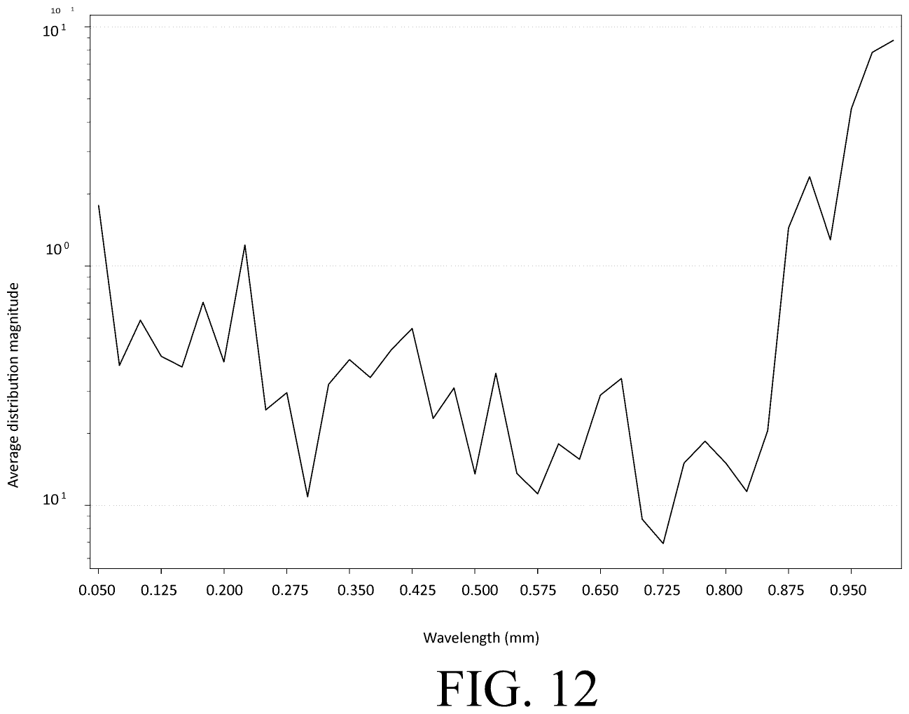

To investigate and validate the empirical feature selection process, an attribution analysis is performed using the method of integrated gradients (see Sundararajan, Mukund, et al., “Axiomatic attribution for deep networks”, 2017 (Sundararajan)) implemented via the Captum package (see Kokhlikyan, Narine, et al., “Captum: A unified and generic model interpretability library for PyTorch: , arXiv e-prints, page arXiv: 2009.07896, September 2020 (Kikhlikyan)). Integrating gradients through the network gives each input feature an attribution score, a signed value representing its effect on the output logits that constitute the classification layer of the network. The cross-validated geometric mean of the attribution magnitude over the test set for each data split is considered as a measure of the influence of a given input feature (i.e. a k-encode) of the network. FIG. 12 shows the geometric mean of the attribution magnitudes for all 39 wavelengths considered in feature selection, computed by integrated gradients for a fully-connected network. The longest wavelengths display the greatest magnitude effect; this is likely due to large wavelengths having strong intensity for areas of the organ without dense stroma, where abnormal growth is unlikely. This is also consistent with the empirical feature selection results, where long wavelengths performed relatively well. The shorter wavelengths display a greater magnitude effect than the central wavelengths.

Leveraging the attribution results for feature selection is considered in two ways: by selecting the top 10 wavelengths by attribution and the top 10 non-adjacent wavelengths by attribution for use as input features. Each of these choices underperformed (in AUC-ROC) the equally spaced set of wavelengths that the experimentation had identified as optimal. This validated the choice of input features.

To investigate and validate our empirical feature selection process, an attribution analysis was performed using the method of integrated gradients (see Mukund Sundararajan, Ankur Taly, and Qiqi Yan. Axiomatic attribution for deep networks. 2017.) implemented via the Captum package (see Narine Kokhlikyan, Vivek Miglani, Miguel Martin, Edward Wang, Bilal Alsallakh, Jonathan Reynolds, Alexander Melnikov, Natalia Kliushkina, Carlos Araya, Siqi Yan, and Orion Reblitz-Richardson. Captum: A unified and generic model interpretability library for PyTorch. arXiv e-prints, page arXiv: 2009.07896, September 2020. Integrating gradients through the network gives each input feature an attribution score, a signed value representing its effect on the output logits that constitute the classification layer of the network. The cross-validated geometric mean of the attribution magnitude over the test set for each data split was considered as a measure of the influence of a given input feature (i.e. a wavelength) of the network. The gradients are computed with respect to the output tumor probability, after the softmax operation.

FIG. 12 shows the geometric mean of the attribution magnitudes for all 39 wavelengths considered in feature selection, computed by integrated gradients for a fully-connected network. The longest wavelengths display the greatest magnitude effect; this is likely due to large wavelengths having strong intensity for areas of the organ without dense stroma, where abnormal growth is unlikely. This is also consistent with the empirical feature selection results, where long wavelengths performed relatively well. The shorter wavelengths display a greater magnitude effect than the central wavelengths.

TABLE S2
Top 5 best-performing subsets of 8 wavelengths
and corresponding AUC-ROC.

| Wavelengths (mm) | AUC-ROC |
|---|---|
| {0.25, 0.35, 0.45, 0.55, 0.65, 0.75, 0.85, 0.95} | 0.776 |
| {0.05, 0.15, 0.25, 0.55, 0.65, 0.75, 0.85, 0.95} | 0.776 |
| {0.15, 0.35, 0.45, 0.55, 0.65, 0.75, 0.85, 0.95} | 0.775 |
| {0.05, 0.25, 0.35, 0.55, 0.65, 0.75, 0.85, 0.95} | 0.775 |
| {0.01, 0.25, 0.45, 0.55, 0.65, 0.75, 0.85, 0.95} | 0.775 |

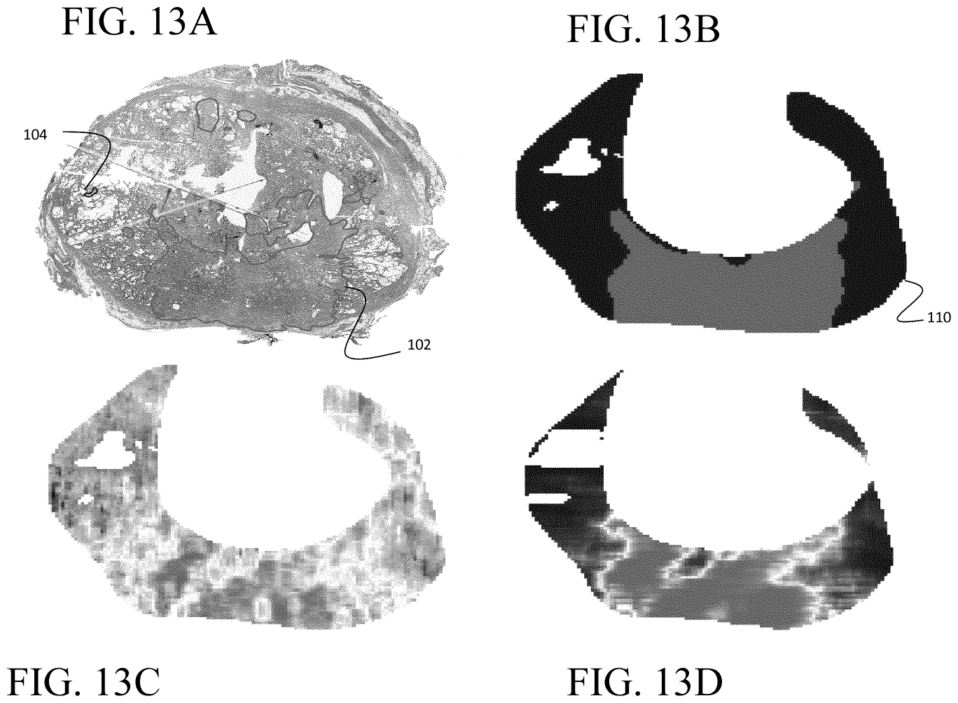

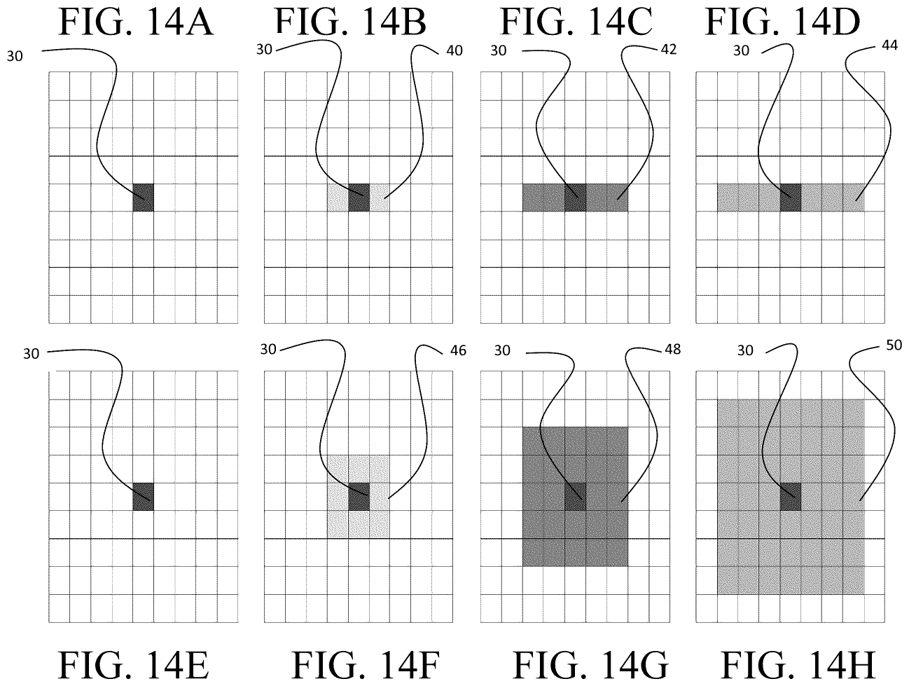

Example model inference results compared to ground truth are shown in FIGS. 13A-13D. FIG. 13A shows an annotated histology slide with a large carcinoma present where (tumor regions 102 red; normal regions 104 blue). FIG. 13B shows a ground truth label map for the peripheral zone 110 as discussed above with regard to FIGS. 10B and 10C; the data acquisition procedure effectively smooths the edges of the carcinoma region. FIG. 13C shows a single-region inference heatmap using a trained neural network. FIG. 13D shows an inference heatmap from a model which incorporates data from neighboring regions, as discussed below, denoising the model predictions.

### Effect of Spatial Information

Because carcinoma manifests as contiguous patches of dense texture generally larger than an individual ROI, it is expected that classification performance can be improved when the model is trained on inputs representing larger areas, thereby providing greater spatial context to each ROI. Rather than expanding the size of the ROIs, which results in washing out important texture signals due to increased variation, the spectrum from each ROI plus the spectra of neighboring ROIs, as previously described, is used as input.

An ancillary benefit of considering a spatially extended set of neighboring ROIs is the ability to estimate the length scale of an identified tumor by making predictions for each ROI in the extended input. This adds another layer to the diagnostic capability of the method, whereby tumors can be both identified and placed within a size class. An algorithm is developed and described for tumor scale estimation as an application of the inferential capabilities of the neural networks as discussed subsequently.

As previously described, to incorporate spatial context, spectra from neighboring regions may be employed. Regions lying on either side of a given ROI is input, along the same axis in the analysis direction, may be used forming a ‘rod’ of inputs. This mimics the clinical procedure in which spectra are measured from multiple VOIs aligned along an excited rod volume FIG. 14A shows the ROI 30 (center red rectangle) in isolation; FIG. 14B the ROI 30 with one nearest neighbor 40 on either side (light blue), giving a (3,1) input size; FIG. 14C the ROI 30 with two neighbors 42 on either side (green), giving a (5,1) input size; FIG. 14D the ROI 30 with three neighbors 44 on either side (yellow), giving a (7,1) input size.

Alternatively, 2D arrays of neighbors in the transverse directions may be employed for square-shaped inputs. FIG. 14E the ROI 30 in isolation; FIG. 14F the ROI 30 with one layer of neighbors 46 in all directions, forming a (3,3) sized input; FIG. 14G the ROI 30 with two layers of neighbors 48, forming a (5,5) input size; and FIG. 14H the ROI 30 with three layers of neighbors 50, forming a (7,7) input size.

Application of rod neighbors for the neighbors in the 2D arrays may also be employed forming 3D arrays of 3,3,3, arrays of 5, 5, 5 and arrays of 7, 7, 7 as previously described.

In practice, arranging the simulated data in rod or square inputs causes a reduction in the overall dataset size, as larger input sizes may extend beyond the edges of the organ. These data are not padded but instead considered only inputs which were fully contained in the organ.

When ROIs are input in aggregate as a rod or square, the classification objective is taken as the label of the central ROI. With a rod input, if n neighbor ROIs are included, the model receives as input 2n+1 spectra, each with 10 k-encode intensities. The training objective is the identification of the label corresponding to only one of these spectra. Throughout, the terminology n neighbors is used to refer to inputs which include 2n+1 ROI spectra in the rod-shaped case or (2n+1)2 ROI spectra in the square-shaped case.

Changing the number of neighbors requires retraining a new neural network in order to account for the change in dimensionality of the input layer. That is, a different network will need to be retrained in order to make predictions on an ROI plus n=1 neighbors, n=2 neighbors, n=3, and so on.

In general, when retraining networks to account for different input sizes, the same hidden architecture is preserved as the best-performing network determined in the prior feature selection parameter sweep, with the only change being the increased dimensionality of the input layer.

### Modalities of Incorporating Neighbor Information

In clinical applications, data acquisition from a rod-shaped set of neighboring ROIs is more efficient than a square-shaped input; MRH measurements naturally excite a rod, whereas measuring an entire square of ROIs requires multiple separate acquisitions. In optimizing the models for the incorporation of neighbor information, rod-based inputs are principally considered.

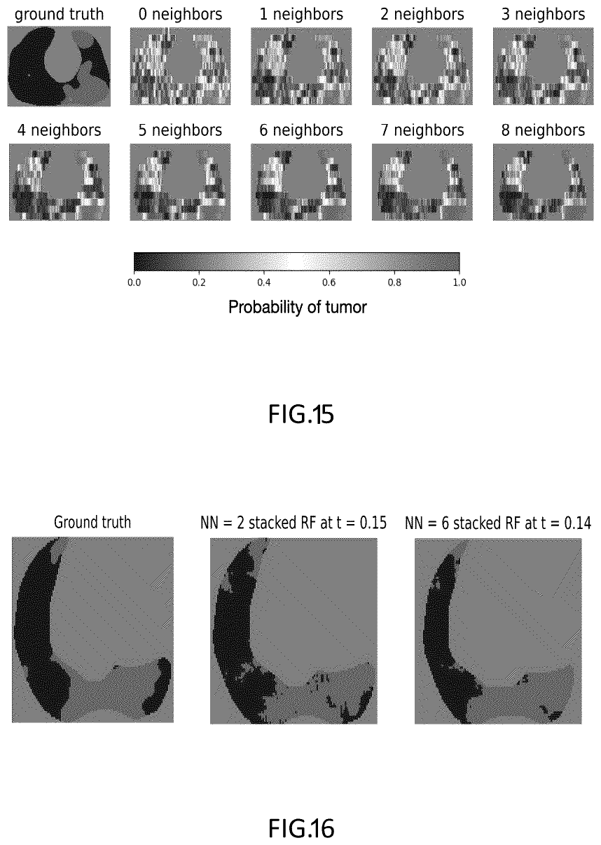

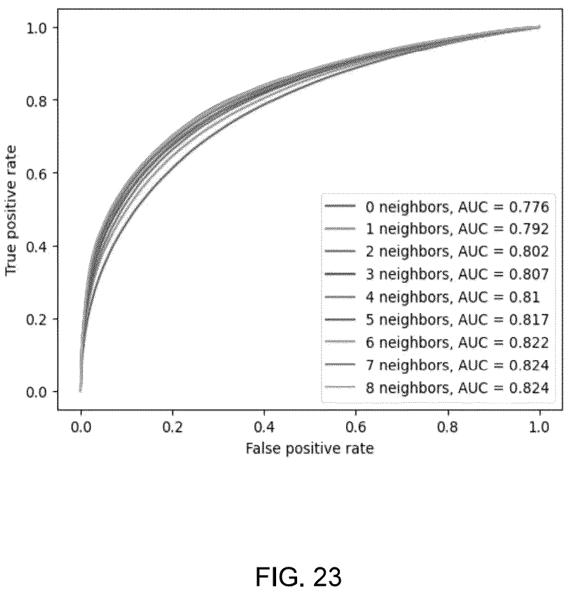

The simplest way to incorporate the neighbor information is to use all spectral intensities as input features to the model in an unstructured fashion, i.e. without any explicit positional encoding. The hope would be that the model learns an implicit notion of locality or learns interrelationships between spectral intensities from neighboring ROIs. FIGS. 15 and 23 show that even when unstructured neighbor information is used as input to a fully-connected network, the models can effectively integrate this information to improve the inferential performance on the central ROI. In a model without neighbors, the network makes predictions with relatively low confidence that the underlying sample is either normal or tumor, as illustrated by the paler blue and red regions, and the white spots scattered throughout the inference map (FIG. 15).

As the number of neighbors increases, the predictions become more confident, as illustrated by the sharpening of the colors and the reduction of the noisy white regions. The predictions become more accurate as well, as shown at the dataset level in FIG. 23; the AUC-ROC increases as the number of neighbors is increases, but with diminishing returns. Indeed, if input sizes are too large, the underlying region will encompass different tissue textures. Performance reaches a peak with input sizes consisting of about 12 neighbors on either side, and declines beyond this size (FIG. 17). This process requires a new network to be trained for every input size 2n+1, which can be expensive. Furthermore, the presence of many unstructured spectral measurements creates an overfitting concern for deep learners.

### Stacked Random Forest

To determine if the models that received unstructured neighbor input were in fact learning complex relationships between neighboring spectra, a new class of models is constructed which by construction did not have access to all the spectral data from neighboring ROIs. Instead, this new class was given only the output of the best-performing fully-connected network for each ROI among the 2n+1 neighbors. The null hypothesis for the experiment was essentially that the models receiving all the spectral data were learning a ‘voting’ process between the tumor likelihoods of neighboring ROIs as opposed to learning more complex relationships between neighboring spectra. The hypothesis is supported if models which by construction can only ‘vote’ perform comparably to models which receive all available spectral data.

A stacked architecture was developed where first, the trained fully-connected network made separate predictions for each spectrum of the 2n+1 input ROIs; this generates 2n+1 target probabilities. These probabilities were used as the input to a random forest (RF) classifier with significant regularization and an objective of classifying the central ROI. An identically configured model is trained for each input size, and the input data is augmented by spatial reversal along the rod to support parity invariance of the predictions.

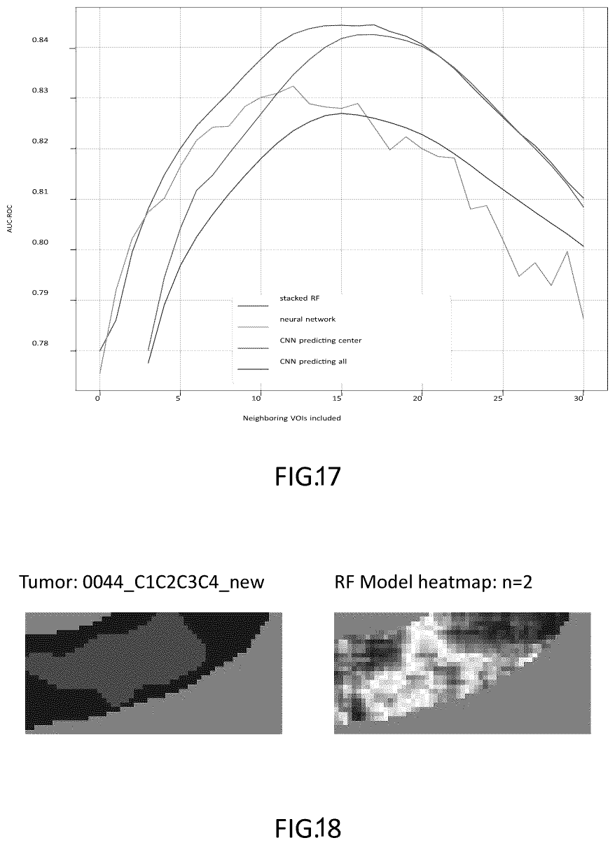

It is found that the stacked RF model performed at least as well as the fully-connected network which received the unstructured spectral intensities from the neighboring ROIs, and at a much lower computational cost in training. The random forest acts as a sort of ensemble voting process on the outputs of the baseline neural network, and the inference results show that with neighboring inputs, the stacked RF model reduces noise and increases model confidence in the same way as the fully connected network; example inference results are shown in FIG. 16. These performance gains, measured by AUC-ROC, can be seen in FIG. 17.

The feature importances of the RF models were also computed to determine which of the 2n+1 inputs were being prioritized. The learned, cross-validated feature importances indicate that the RF models did learn a notion of locality: central ROIs had greatest relative importance which became attenuated with distance from center. An additional increase in feature importance was observed at 8 ROIs from center, which is the point at which the data acquisition window does not overlap the central prediction ROI.

In the stacked RF model, the RF component has no access to the spectral data by construction. The fact that it performs as well as the fully-connected network which receives all the spectral data suggests that the fully-connected architectures were not featurizing across neighboring spectra, or at least were hampered by overfitting. The stacked RF models were the best-performing architectures for incorporating neighbor information, and were used for the tumor size inference problem addressed later in this work.

### Convolutional Neighbor Model

Given any spatially-structured data like a rod or square-shaped input, the natural choice for translation equivariant featurizing layers is convolutions. An alternative convolutional neural network model was developed which predicts every label in its 2n+1 ROI input. It was found that, evaluated by AUC-ROC on all its outputs, the CNN model underperformed the stacked RF models across input shapes, shown in FIG. 17. Comparing the CNN, which predicts all the labels of the input, to the RF models, which predict only the central ROI label, is not necessarily meaningful. When comparing the two only on the basis of their prediction of the central ROI, the performance converges and is equivalent for large input sizes n≥16, but the CNN's predictive performance is still weaker for smaller input sizes. Despite the gap in performance, the CNN model has significant advantages over the stacked models. Principally, it predicts the labels of all ROIs in the rod, and it can do so for any input with n≥3; the lower bound is at the convolutional kernel size. Because it can accept different-shaped inputs, the same trained model can be used for inference across input shapes, whereas a different RF model must be trained for each value of n.

### Tumor Size Inference-Determining Tumor Size

It is of great clinical interest to leverage the classification models developed in this work to not only identify certain ROIs as cancerous, but to also obtain an estimate of tumor size. If data is acquired in a rod of neighboring ROIs, an estimate of linear tumor size can in principle be extracted from the classification results.

A reliable estimate of tumor size over all size classes should involve collaboration of models with differing input sizes; when model performance is analyzed on data segregated by tumor size, models with spatially extended inputs tended to underperform in their detection of small tumors. Using an ensemble of models which accept different amounts of spatial context equalizes performance across tumor size classes. Each model, with a specific input size, will also have an associated optimal classification threshold which balances precision and recall. In the size inference algorithm, a small range of thresholds around optimality is used in order to obtain predictions which emphasize both precision and recall. With these points in mind, an algorithm was sought which, given multiple adjacent rod-shaped inputs, can provide an estimate of tumor size along with confidence bounds around that estimate.

An experiment is carried out using the aggregated stacked RF model and the baseline fully-connected neural network. Input shapes of 2n+1 ROIs with 0≤n≤16, with the upper bound chosen at the approximate point of performance saturation as measured by AUC-ROC are considered. For a given data split, the model and the associated test set is loaded, which contains three or four organs. In a given organ slice, tumors are identified and a local sub-array containing the tumor is selected. The sub-array may be viewed as a set of adjacent rod-shaped inputs on which inference may be performed to generate a heatmap of model predictions. An example of an isolated sub-array containing a tumor and the associated inference heatmap is shown in FIG. 18.

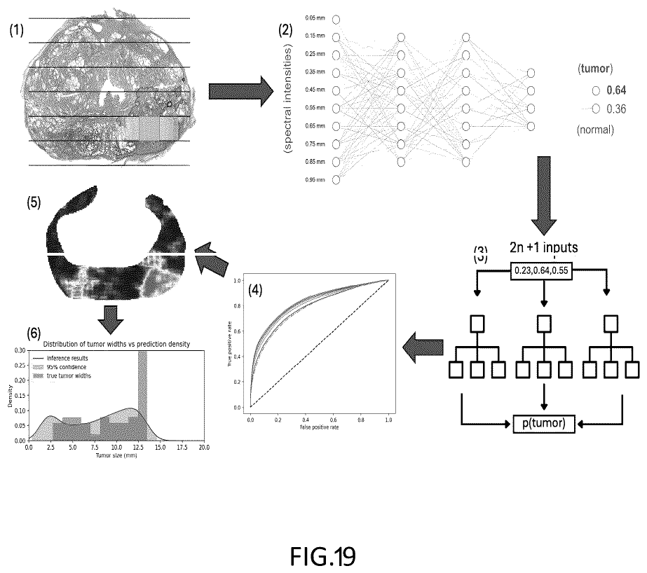

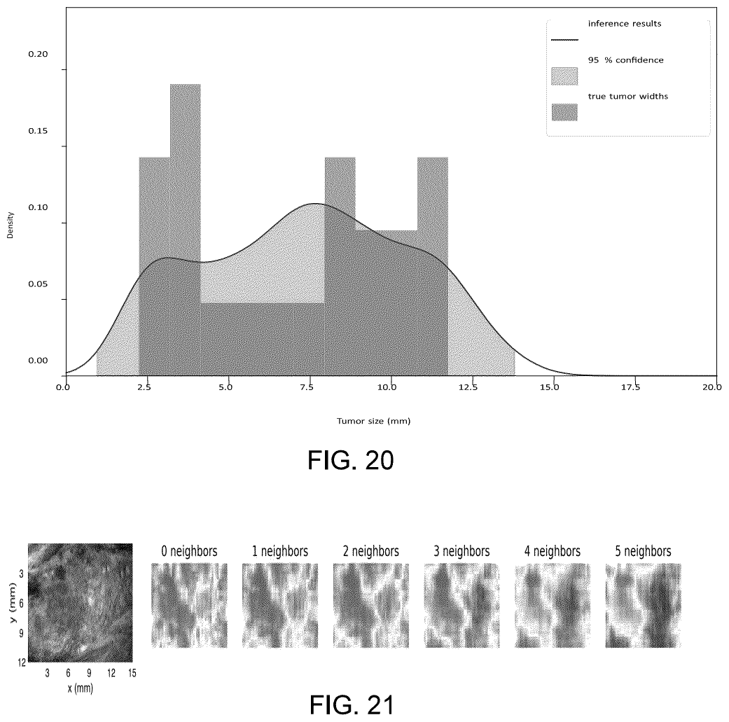

A tumor size estimation algorithm is developed, an overview of which is shown in FIG. 19. The technical details of the algorithm are described subsequently. The method shown in steps 1-6 obtain a set of size estimates which are aggregated over different models as well as different classification thresholds. Kernel density estimation (KDE) is done on the collection of estimates to obtain the final result. Performing the size estimation algorithm on the sub-array pictured in FIG. 18 gives a density function over tumor sizes. A confidence interval can be computed from this density function by the log-likelihood method or Fisher information-related metrics. This density is compared to the distribution of label widths of the ground truth tumor sub-array. A comparison of the size inference and the labeled tumor widths is shown in FIG. 20.

The density function agrees with the actual tumor widths, and that the confidence interval correctly contains the actual tumor widths from the label sub-array of FIG. 18. The dispersion in the estimate introduced by the use of multiple thresholds and multiple input shapes, in addition to the KDE process, gives a sense of the spread of the size distribution and therefore the model's confidence. Clinically, only a few rod-shaped inputs may be available for analysis. In this case the development of dispersion in size prediction through the threshold range and model ensembling is even more important to give an accurate estimate and sense of confidence in the size estimate.

### Inference of High-Resolution MRI Data

An application of the model to a limited test dataset that more closely represents the data acquired in clinical practice is demonstrated. The test dataset consists of ex vivo prostate segments imaged with high-field (7T) MRI at 60×60×90 μm resolution, selected from the data previously analyzed in Durand, Matthieu, et al., “Magnetic resonance microscopy may enable distinction between normal histomorphological features and prostate cancer in the resected prostate gland”, BJU International, 119(3): 414-423, March 2017 (Durand). That is, each image slice is 90 μm apart, over an order of magnitude smaller than the approximately 3000 μm separation of the histology slices used in the training dataset.

From the 3D MRI data, a contiguous region 12 mm×15 mm×2 mm in the center of the organ is extracted. This region is further divided into 3D VOIs that are 2 mm×2 mm×2 mm in size. The VOIs are collapsed along the last two dimensions, leaving a 2 mm-long 1-D signal from which a spectrum is generated at wavelengths between 0.15 and 0.95 mm, in increments of 0.1 mm. This matches the wavelengths used to train the neural network described above, with the exception of 0.05 mm (which would not be achievable since the resolution of the 3D MRI data is 0.06 mm along the analysis dimension). The neural network is retrained on these 9 wavelengths extracted from the spectra generated across the 114-slice training dataset. The network is then tested on the spectra of the 3D MRI images. Because the 3D MRI data is not annotated, ground-truth labeling is not available; however, illustrated here is a prediction map of the likelihood that each VOI contains tumor tissue (FIG. 21). Generating the prediction map for the models trained on 1 up to 5 neighbors on either side of the VOI in a rod is repeated. Without neighbors, the model predicts a high prevalence of cancer across the sample. As the number of neighbors is increased, the region predicted to be tumor shrinks, but remains consistently in the right half of the sample. Similar to what is observed when neighbor information is incorporated in the original dataset, as shown in FIG. 15 the probability values for normal tissue sharpen as the number of neighbors increases, with more confident prediction probabilities.

### Discussion

As disclosed herein, a sub-millimeter-resolution textural data acquisition method called MRH (magnetic resonance histopathology) is introduced and an in silico validation of its potential in identifying prostate cancer is performed for estimating the size and severity of tumors. MRH trades off image acquisition for a measurement of texture that can achieve higher resolution more comparable to scale at which glandular structure varies. The textural signature of tissue is observed to change with the onset of disease (Barrett). Here, deep learning classifiers are trained on simulated MRH data to demonstrate how texture measurements can be used to differentiate between healthy and cancerous tissue.

MRH procedures can be added alongside standard multi-parametric MRI prostate scans to obtain an additional dimension of information for determining cancer likelihood and severity. This enables more accurately targeted biopsy, reducing patient discomfort and risk. During MRH acquisition, a single spatial frequency is measured in each excitation, with multiple spatial frequencies measured over repeated excitations. While exact spatial coherence is unnecessary to measure texture, it is still important to minimize patient motion between acquisitions and limit acquisition time. Hence, a critical challenge is choosing an optimal number of spatial frequencies to measure in order to obtain enough diagnostic information in as little time as possible. Here, a feature selection procedure and subsequent attribution analysis are performed which demonstrate that 10 wavelengths, evenly sampling a spectrum between short (0.05 mm) and long (1 mm) length scales, best captures textural variation.

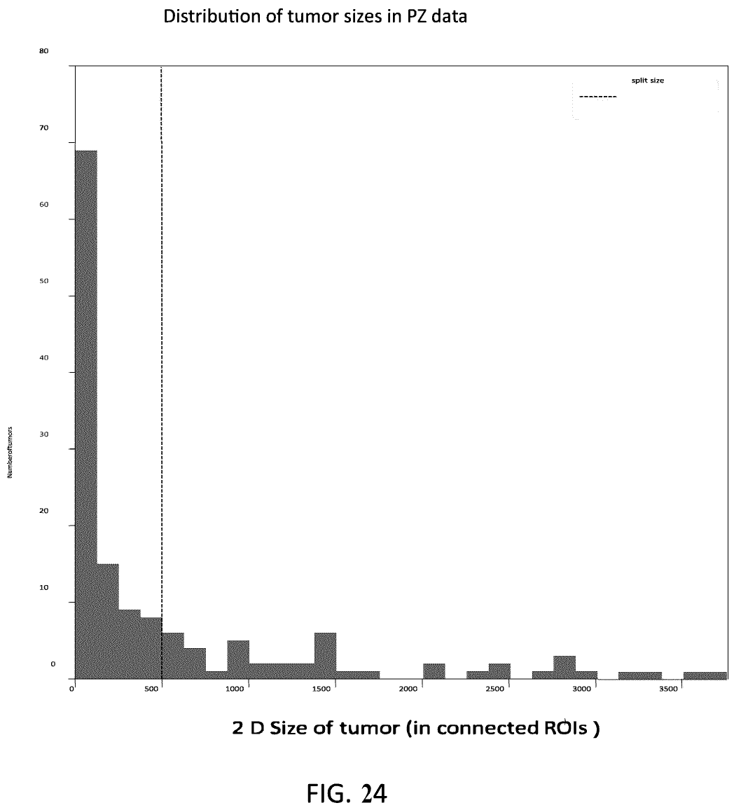

While the model performs well when trained on samples treated as isolated and uncorrelated in space, since tumors are largely contiguous, approaches to incorporating spatial context that improve performance and de-noise results are demonstrated. Models are created that make predictions using samples alongside their n nearest neighbors. It is found that increasing the number of neighbors can enable more accurate and more confident predictions, up to a maximum, at which the input underlies too much spatial variation. These models also enable estimating distributions of the sizes of predicted tumors. In practice, the resolution of this distribution will be set by the density and width of rods of tissue that are excited during the series of MR measurements. Given the good agreement between predicted and actual tumor size distributions (FIG. 24), even with a sparse sampling, it is expected meaningful estimates of tumor size can be obtained. When combined with MR imaging of lesions, MRH can validate and enhance the characterization of tumors, and set guidelines for further biopsy. In this manner, several targeted MRH acquisitions, used to generate estimates of tumor size and severity, can function as a noninvasive analogy to biopsy.

The higher resolution of MRH can also enable more detailed grading of lesions akin to the Gleason scoring method used in histopathology. Currently, the PI-RADS standard for MRI involves scoring each lesion on a scale of 1-5 indicating the likelihood of clinically significant cancer (Turkbey). MRH trained on pathologist-graded histology could be used to not just determine the likelihood of cancer, but to grade its severity as well. In future work, models can also be trained not only on data containing tumors labeled over a range of grades, but on other non-cancerous but atypical cases such as prostatic intraepithelial neoplasia.

The study focused on simulated MRH measurements in the peripheral zone, from which approximately 70% of tumors originate. However, over 20% of tumors originate in the transition zone, and as such it is critical to expand analysis outside the PZ. Identifying tumors in the transition zone remains challenging with mpMRI, since benign hyperplasia and stroma in this region can be confused with cancer, leading to false positives (see Kitzing, Yu Xuan, et al., “Benign Conditions That Mimic Prostate Carcinoma: MR Imaging Features with Histopathologic Correlation”, RadioGraphics, 36(1): 162-175, January 2016. Publisher: Radiological Society of North America (Kitzing). The increased resolution of MRH can allow further training on tissue outside of the PZ and potentially aid mpMRI in resolving confusion between benign and cancerous tissue. In the disclosed experimentation, it was found that model precision and overall performance suffered when training on entire-organ data: many areas of dense stroma outside the PZ were falsely flagged as cancerous. Most likely, different models should be used for PZ and non-PZ parts of the organ to avoid this type of confusion. A separate, auxiliary classifier could be developed to determine whether a given MRH measurement lies within or without the PZ, though in a clinical setting this information is likely known ahead of time. With the focus of developing MRH analytics only for tumor detection, the problem of classification outside of the PZ was considered as beyond the scope of the present work.

### Methods

### In Silico Analysis of Prostate Images

MRH measurements are simulated from “ground truth” histology images that are of much higher resolution than what in vivo (and in some cases, ex vivo) 2D MRI can achieve. These slides are obtained from the dataset described in Singanamalli et al., and consist of 114 digitized H&E stained slides obtained from 16 excised prostates. Each organ is divided into 3-4 mm slices, and then segmented into 4 quadrants per slice. Each quadrant is imaged at 0.5 μm resolution, and the quadrants are then stitched together to form a full slice image using the Histosticher software (see Chappelow, Jonathan, et al., “HistoStitcher (C): an interactive program for accurate and rapid reconstruction of digitized whole histological sections from tissue fragments”, Comput Med Imaging Graph, 35(7-8): 557-567, 2011 (Chappelow)). The slices are annotated by a pathologist, with regions labeled as CAP (carcinoma of the prostate), PIN (prostatic intraepithelial neoplasia), or atypical (FIG. 10A). In the following analysis, only carcinoma annotations are used to determine positive labels (FIG. 10B).

To improve model performance, the dataset is restricted to include only the peripheral zone (PZ) of each prostate. Most tumors originate in the PZ, with an incidence of about 70% (see McNeal, John E., et al., “Zonal Distribution of Prostatic Adenocarcinoma: Correlation with Histologic Pattern and Direction of Spread”, The American Journal of Surgical Pathology, 12(12): 897, December 1988 (McNeal)), and most of the tumors in the dataset are contained within the PZ. Moreover, excluding other regions allows us to avoid dense stroma that can be easily confused with carcinoma; the dense stroma is typically localized to known regions that can be excluded from clinical MRH data acquisition, avoiding confusion in the diagnostic process. The PZ of each image slice (see Fine, Samson W. and Reuter, Victor E., “Anatomy of the prostate revisited: implications for prostate biopsy and zonal origins of prostate cancer”, Histopathology, 60(1): 142-152, 2012 (Fine)) is manually segmented and only this segmented region is used to train the models (FIG. 10C).

### Simulating MRH

MRH acquires a high-resolution 1D signal; however, a 3D volume must be excited to measure this signal since it is impossible to excite a 1D line. First, a slice-selection gradient and 90-degree pulse is applied to excite a slab of tissue. Then, a gradient in an orthogonal direction and 180-degree refocusing pulse is applied to excite a slab orthogonal to the first. This defines a rod formed by the intersection of the two slabs. Volumes of interest (VOIs) along this excited rod can then be spatially encoded in the same TR. In each VOI, the direction of the k-encode, referred to as the analysis direction is orthogonal to the long axis of the rod. The result is a 3D volume that gets “smeared” in the two dimensions orthogonal to the analysis direction to effectively provide a 1D signal. This process of exciting a rod and k-encoding VOIs is repeated multiple times to obtain signals from a large volume of tissue. In practice, the VOIs will be approximately 4 mm long in the analysis direction, which is aligned with the head-foot axis (parallel to the urethra), and 2.5 mm×2.5 mm along the remaining two dimensions.

To simulate MRH spectral data, 2D regions of interest (ROIs) are selected from ground-truth histology images of an organ. However, the dataset consists of high resolution images of the histology slide, in the example case, images of a prostate taken perpendicular to the direction of the urethra. When simulating MRH data, any analysis direction chosen for these ROIs must lie within the plane of the image and will thus be orthogonal to the clinical analysis direction. The assumption is made that the prostate texture is roughly isotropic, and that the simulated data can be analyzed analogously to clinical data.

Each image is converted to grayscale and divided into 2 mm×2 mm regions of interest (ROIs), corresponding to 1000×1000 pixels (FIG. 10D). Each ROI is collapsed along one direction, labeled the “transverse direction” giving a 1D signal in position space along the analysis direction. In the disclosure herein, the transverse direction is chosen to be parallel to the sagittal direction, and analysis direction to lie along the coronal direction.

Since each full image of a prostate slice is of order (2·104)×(2·104) pixels, this would provide about a 20×20 grid of 400 ROIs total; however, several of these will lie outside the PZ or outside the boundaries of the prostate, and the amount of usable data will be relatively small. Moreover, the ability of a model to classify a given sample may depend on how precisely positioned that sample is within a tumor. To expand the dataset, the image is divided into a grid of overlapping ROIs with stride 0.25 mm. It is also noted that in clinical practice, the spatial encoding of an excited rod can result in encoded VOIs that overlap with one another in space.

During an MRH procedure, the MR scanner signal measures the similarity of the texture of the excited tissue to the encoded k-value, or wavelength. Repeated k-encode measurements then construct a spectrum of intensities over a range of wavelengths. This spectrum is simulated on the histology slices by performing a (discrete) Fourier transform on the 1D, averaged ROI over a set of specific k-values. The frequency-space spectrum generated by this process is representative of the clinical measurements made in an MRH procedure, and is used to train the deep learning models.

A simulated MRH spectrum can be computed at any wavelength between 0.004 mm and 1 mm, as per the Nyquist theorem. However, in practice, MRH can achieve wavelengths only as small as approximately 0.05 mm. Thus, the spectrum is first computed at 39 wavelengths between 0.05 mm and 1 mm in 0.025 mm increments—this spectrum is a much higher resolution than what would be achieved clinically. In the subsequent section, the feature selection process is described, in which it is determined which subset of 10 wavelengths is most informative for classification, and whether the model's performance is significantly hampered by using few wavelengths compared to the more detailed spectrum, thereby informing parameters for MRH k-encodes.

### Neural Network Classification

Deep neural networks are trained on simulated MRH spectra. The problem is one of binary classification given the intensities of the input wavelengths for a single ROI. In the experimentation, a wide array of models and architectures is considered. Each model was tested on the same dataset with identical cross validation splits and performance was compared on the basis of the AUC-ROC score. Classical ensemble learners like random forests and gradient boosting trees underperformed deep learning models even after hyperparameter optimization. Transformer (attention-based) models were considered, adapting the architecture disclosed in Liu, Ze, et al., “Swin transformer: Hierarchical vision transformer using shifted windows”, 2021 (Liu) for the multihead self-attention blocks; these were comparable but slightly subpar in performance to simpler and shallower fully-connected neural nets. Also considered were one-dimensional featurizing convolutions along the spectrum, with the idea that local correlations between similar wavelengths could be effectively featurized for prediction, but these models also did not perform well. Fully-connected networks were the focus to classify individual ROIs by their input k-encode intensities.

### Feature Selection

In the in silico analysis, spectra can be obtained from each ROI sampled at a higher number of wavelengths (k-values) than would be typically achieved in a clinical k-encoding procedure. While simulated spectra are computed at about 40 wavelengths, from 0.05 mm to 1 mm in increments of 0.025 mm, a clinical MRH procedure would involve approximately 10 k-encodes to limit the length of the scan and acquire more samples from a wider region of tissue in the same amount of time. The goal is to find a relatively small subset of k-values which maintain diagnostic power while reducing acquisition time. To study this trade-off, a feature selection routine is performed to determine which wavelengths are most informative for binary classification.

A comparison is provided of the performance of neural network classifiers each trained on 5 sets of different inputs, each containing 10 features: the spectral intensity at a) short wavelengths (between 0.05 and 0.275 mm, in increments of 0.025 mm); b) medium-short wavelengths (between 0.3 and 0.525 mm, in increments of 0.025 mm); c) medium-long wavelengths (between 0.55 and 0.775 mm, in increments of 0.025 mm); d) long wavelengths (between 0.775 and 1 mm, in increments of 0.025 mm); and e) wavelengths spanning the entire range (between 0.05 and 0.95 mm, in increments of 0.01 mm). In each case, the spectral intensities in the train and test datasets were globally normalized to zero mean and unit standard deviation, appropriate for the zero-centered activation functions in the neural network. These results trained on a subset of input features are also compared against a classifier trained on all 40 wavelengths as a (clinically infeasible) benchmark and upper bound on predictive performance.

As a feature selection experiment a joint parameter-hyperparameter sweep is performed to determine optimal architectures and input features. For each set of wavelengths, multiple fully-connected network architectures are tested. Each network architecture consists of the input layer, at least 1 hidden fully connected layer with GeLU activation followed by batch normalization and dropout, and an output layer with 2 output nodes, which, after undergoing a softmax operation, give the probabilities of a sample as being normal or tumor. If there are multiple hidden layers, the last hidden layer contains half the number of nodes of the preceding layer. For each set of features, architectures are tested arising from all combinations of the following parameters: {1, 2, 3} hidden layers, of dimensions {16, 32, 64}, with dropout probabilities of {0, 0.1, 0.2, 0.5}. Details of the model configurations and training procedures are included in Appendix A.

For each configuration (set of features and network architecture), a 5-fold cross validation procedure is performed. The data is first sorted by the organ from which it was obtained (a total of 16 organs). All data from 3 to 4 organs—comprising approximately 20% of the total dataset—is withheld for testing, and the model is trained on the remaining data; splitting by organ is crucial to avoid leakage between train and test sets. This procedure is repeated for a total of 5 folds, ensuring that all data has been seen in the training set.

With the 10 evenly spaced wavelengths as the input features, the best-performing architecture for single-spectrum classification was the fully-connected network with layers (64,64,64,32) into two output logits, with batch normalization and dropout (p=0.05) after each hidden layer. This architecture achieved a cross-validated AUC-ROC of 0.780. At the optimal classification threshold as determined by the F1 score, the (cross-validated) model had 82% accuracy, precision 44%, and recall 52% on the test set. This fully-connected model, trained on 10 wavelengths, formed the backbone for most of the inferential analysis. Incorporating neighbor information

The MRH measurement process naturally involves exciting a rod of tissue from which k-encode measurements can be made in a localized region of the organ rather than strictly a single VOI. Carcinomas, being localized, generally contiguous objects, should theoretically be easier to identify if their local context within the organ is known. This provides a basis for incorporating multiple neighboring spectra into the input to the predictive models.

Inputs with n neighbors contain (2n+1) spectra. Since the simulated MRH measurements are made with overlapping windows, an input with n neighbors covers a real spatial extent of 2+0.5n millimeters in the organ. Neighbors are included symmetrically about the central ROI from which the classification label is drawn.

Three architectures are considered which incorporated the spectral information from neighboring ROIs: a fully-connected network, stacked random forest models, and a convolutional neighbor model. Details are included of these models' configurations and training processes are provided subsequently. Regardless of the modality, incorporating neighbor information cleaned up predictions and improved the performance of models as evaluated by the test AUC-ROC, recall, and precision. This supported the hypothesis that local context was important for correctly identifying tumors. FIG. 17 shows how the AUC of each model increases with the number of neighbors being input before saturating and decreasing thereafter. Above, in FIG. 23, the ROC curves are included for the neural network trained with neighbor input, where the performance improvements are clear.

However, some inferential results showed that models with large inputs containing many ROIs may have become less sensitive to smaller tumors. The reason for this is straightforward: as the size of the input extends beyond the spatial extent of the tumor, ‘normal’ spectra far from the central ROI may influence the model to produce a negative prediction. Without any positional encoding, how a model determines which spectra corresponds to the label is up to the training process, and may result in mis-prioritizing spectra relative to the center of the region considered. While this is not an issue for large, extended tumors, and likely even improves model recall, the lack of explicit positional encoding or a hard-wired notion of locality can confound model predictions for large inputs containing a small, highly localized carcinoma signal.

### Performance on Tumor Size Classes

In the (peripheral zone segmented) histology dataset used for this work, there is a variety of annotated tumor sizes ranging from only a few ROIs across to tumors which extend to cover a macroscopic fraction of the organ slice under consideration. Each tumor was clustered and its size computed, defined as the number of connected neighboring ROIs that constituted the tumor. Pictured in FIG. 24, the distribution of sizes is heavily skewed, with many small tumors of size <100 ROIs and a long tail of large tumors comprising 1000 ROIs or more. By nature, the large tumors comprise a much larger part of the dataset despite being a minority in the distribution of sizes.

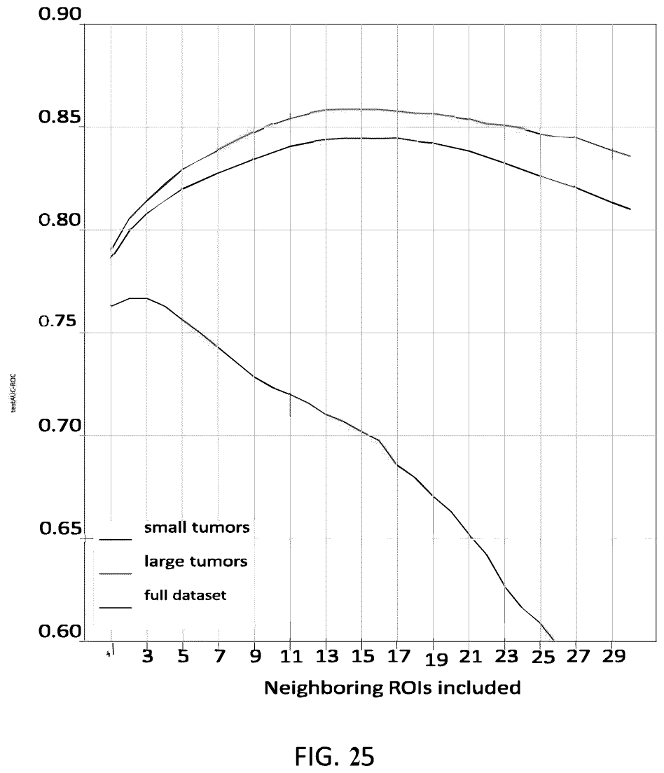

The dataset is split between small tumors (comprising <500 connected ROIs) and large tumors. The non-tumor data is equally and randomly split, resulting in two datasets of roughly equal size for each tumor class. To examine the effect of tumor size on the inferential results, the trained stacked RF models on each split dataset are re-evaluated and the metrics compared. The results, shown in FIG. 25, demonstrate that the inclusion of many neighboring ROIs increases the performance on large tumors while diminishing performance on small tumors. This suggests that in practice, while neighbor information generally cleans up predictions, one must strike a balance between overall precision and sensitivity to small tumors.

### Tumor Size Inference

With data gathered in rods of ROIs, predictions can in principle be made for a spatially extended region of the organ. While most of the models which incorporate neighbor information aim to predict the central VOI with higher confidence, by combining these models an estimate of tumor size can be determined by predicting each ROI along the rod. Here, the meaning of tumor size is an estimate of linear tumor scale along the rod where measurements are made. If multiple rods of ROIs are excited, these linear size predictions can be combined to give an estimate of the overall area of the cancerous region. This process is largely independent of the details of the underlying predictive models.

The simplest way to estimate tumor size is to choose an input shape, perform inference, and threshold the target probabilities. The estimated size in each row is then simply the number of positive predictions in each row. However, this procedure does not give any dispersion to the estimate and does not incorporate the varying strengths of the models with differing input shapes. The size predictions are ensembled in two ways: by combining the estimates of models with different input shapes and by modulating the classification threshold.

For each row of the sub-array, input shapes are constructed of size 2n+1 for n≤16, if possible, or as large as the sub-array allows. Each sub-model in the aggregated stacked RF model generates an inference heatmap using inputs of appropriate size. The inference heatmaps are then thresholded for a range of classification thresholds designed to generate a confidence interval of size predictions.

While multiple methods for determining optimal classification thresholds exist, the focus herein is on the maximization of the F1 score, the harmonic mean of precision and recall. The F1(t) score, a function of classification threshold t, is maximized at some optimal threshold t*∈[0,1]. Because of the classical bias-variance tradeoff, the curve F1(t) is generically convex in a neighborhood of this point.

A range of thresholds can be generated around the optimal t* in a natural way by considering the second derivative of F1(t); this defines a natural scale of curvature of the F1 curve around t*. This length scale will be small for models highly sensitive to the classification threshold and large for models with less threshold sensitivity. An interval of thresholds is defined around optimality using this length scale as [t*−L_{F1}/2,t*+L_{F1}/2]. These perturbed thresholds will emphasize inferential recall when below the optimal threshold and emphasize inferential precision when above the optimal threshold. In doing this, advantage is taken of each side of the precision-recall tradeoff when aggregating the inferential results.

### Size Estimation Algorithm

The algorithm for size inference can therefore be summarized as follows. A tumor sub-array is isolated and each row is divided into rod-shaped inputs of shape 2n+1 with 0≤n≤16. Inference is performed with the appropriate module of the stacked RF model for each input shape, generating inference heatmaps for each value of n. For each input size, a fixed number of thresholds Nt is generated in the interval [t*−L_{F1}/2,t*+L_{F1}/2] defined by the F1 curve. For each index i, 1≤i≤Nt, a predicted (thresholded) label array is generated across all rows and input shapes and averaged across input shapes. In each row, the size estimate sᵢ is computed as the sum of the values of the (averaged) predicted label array. This size estimate is converted to physical millimeters from ROIs.

Carrying out this procedure across all Nt thresholds gives Nt size estimates for each row of the input array, on which a Gaussian kernel density estimation is performed to obtain a density function over physical tumor sizes in millimeters. This density function can be used to compute statistics on the predicted tumor size in the sub-array.

### Model Configurations

### Fully Connected Neural Networks

The fully-connected neural network architecture consisted of a variable number of hidden layers and an encoding output block with two output channels. Probabilities were generated by applying a softmax to the model output. Each hidden layer was followed by a batch normalization (see Ioffe, Sergey and Szegedy, Christian, “Batch Normalization: Accelerating Deep Network Training by Reducing Internal Covariate Shift”, arXiv e-prints, page arXiv: 1502.03167, February 2015 (Ioffe) and dropout block as described in Srivastava, Nitish et al., “Dropout: A simple way to prevent neural networks from overfitting. Journal of Machine Learning Research, 15(56): 1929-1958, 2014 (Srivastava), which is found to greatly improve training stability. The Adam optimizer, as described in Kingma, Diederik P., and Ba, Jimmy, “Adam: A method for stochastic optimization. arXiv preprint arXiv: 1412.6980, 2014 (Kingma), is used with a learning rate of 0.0001 (with cosine annealing) and weight decay, and the class-weighted binary cross-entropy loss. Improvements were not seen when using focal or Dice loss during training. Experimentation with Gaussian white noise data augmentation was also used, but this generally reduced model performance.

When incorporating neighbor information to the fully-connected networks, the data was first generated in structured arrays of 2n+1 10-wavelength spectra for inputs with n neighbors to the central ROI. The prediction objective was the central VOI label. The data was flattened to form 10·(2n+1) input features. No structure or positional encoding was given to these inputs.

### Stacked Random Forest Models

The stacked RF models were trained on the same train/test splits as the best-performing fully connected network for label prediction from the spectrum of a single ROI. On each data split, the trained baseline neural network performed inference on the data and generated a collection of (2n+1) probabilities for each rod of ROI inputs along with the associated central label. Each rod was augmented by its spatial reversal, which did improve model performance.

Random forest classifiers were trained with 200 estimators, slight alpha regularization at 1e-4, max depth 10, and a square root decrease in the number of features considered at each node. These classifiers were evaluated identically to the neural network models. The random forests tended to have less classification threshold sensitivity than the fully-connected networks which accepted neighbor input.

### Convolutional Neighbor Model

The convolutional model architecture consists of three one-dimensional convolution layers with residual connections followed by a number of self-attention blocks and a final two-layer fully-connected classification module A large kernel size of 7 ROIs was found to perform best, and the model objective was set as the prediction of all the labels in the 2n+1 ROI input, where n is the number of neighboring ROIs. This objective is different from the stacked RF models which only predict the central ROI. After a hyperparameter search, a CNN was trained with three convolutional layers (32,64,64), three (singlehead) attention blocks, and a final fully-connected output block (16,8,2) on inputs with 16 neighbors (33 ROIs); this is a real spatial extent of 1 cm in the organ as the input. ReLU activations were used, batch normalization after the convolutions, and dropout with p=0.3. The training procedure was essentially identical to that used for the fully-connected networks.

While this model for size inference is not disclosed herein, it is included for completeness and to demonstrate consideration of traditional image segmentation architectures (there is no obstacle in principle to using any architecture for the size inference procedure). Because the dataset has many channels compared to traditional image data and is generally only available in small subarrays, techniques that perform well in binary or semantic segmentation of images are not necessarily suited to the present task, where large-scale features are generally inaccessible.

Having now described various implementations in detail as required by the patent statutes, those skilled in the art will recognize modifications and substitutions to the specific embodiments disclosed herein. Such modifications are within the scope and intent of the present invention as defined in the following claims.

1. A method for training a neural network for analysis of MRH data, the method comprising:
selecting a plurality of 2-dimensional regions of interest (ROI) in each of a plurality of histology images or electronic images of a cancerous organ, each image annotated with normal and tumor regions indications;
collapsing each ROI in the plurality of ROIs in a transverse direction associated with a transverse width of the ROI to produce a 1 dimensional signal in an analysis direction associated with an analysis width;
convolving the 1 dimensional signal in each ROI with a set of complex sinusoidal functions to compute a simulated MRH spectrum as a function of wavelength;
computing simulated spectra from each ROI in the plurality of ROIs over a plurality of k-values producing a plurality of training datasets corresponding to each image in the plurality of images;
selecting one or more neighborhoods of each ROI from a selected plurality of neighboring ROIs and adding the spectra of those neighbors to the training dataset of that ROI;
receiving the simulated spectra from each ROI in a neural network to produce a two-element probability vector that the input spectrum for that ROI is from normal tissue and the probability that it is from tumor, with these values summing to 1, training the network by comparison of the resulting probability vector with the normal and tumor regions indications annotated for that ROI.

2. The method as defined in claim 1 wherein the selected plurality of neighbors comprises the set of 4 nearest neighbors, 8 nearest neighbors, 24 nearest neighbors or 16 non-adjacent neighbors.

3. The method as defined in claim 1 wherein the step of selecting a plurality of 2-dimensional ROI comprises selecting rods of a fixed width comprising two dimensional columns with an imposed width in the third dimension in ground truth images as a simulation of volumes of interest (VOIs) in actual MRH data wherein the width of the selected rods correspond to the analysis width and dividing the rods crosswise into ROIs.

4. The method as defined in claim 1 wherein the plurality of images comprise whole-organ histology slides imaged and annotated by a pathologist with normal regions and tumor regions.

5. The method as defined in claim 1 wherein the plurality of images comprise pseudo whole-organ digitized H&E-stained histology slides, with annotations indicating tumor, prostatic intraepithelial neoplasia (PIN), and atypical glands with the tumor annotations converted to an image mask used to label the regions of interest (ROI) as normal or tumor and the whole-organ slide is segmented into a peripheral zone divided into the ROIs.

6. The method as defined in claim 1 wherein the plurality of images comprise a plurality of MRI image slices derived from an MRI dataset and annotated by a pathologist with indications of tumor or normal tissue to create a ground truth labeled dataset.

7. The method as defined in claim 1 wherein ROIs containing tumor samples are assigned a higher class weight than normal samples, defined as the inverse of the prevalence of tumor samples in the training dataset.

8. The method as defined in claim 1 further comprising:
shuffling the training datasets and splitting the shuffled datasets into two portions;
receiving the simulated spectra from each ROI in a first one of the portions and the selected neighborhood of that ROI in the neural network to produce a two-element probability vector that the input spectrum for that ROI is from normal tissue and the probability that it is from tumor, with these values summing to 1, training the network by comparison of the resulting probability vector with the normal and tumor regions indications annotated for that ROI;
receiving the simulated spectra from each ROI in a second one of the portions and the selected neighborhood of that ROI in the neural network to produce a two-element probability vector that the input spectrum for that ROI is from normal tissue from a first of the two elements and that it is from tumor from a second of the two elements, with these values summing to 1 to validate the training of the neural network on the first portion by comparison of the resulting probability vector with the annotated normal and tumor regions indications for that ROI.

9. A method for training a neural network for analysis of MRH data and employing the neural network to analyze MRH data, the method comprising:
selecting a plurality of 2-dimensional regions of interest (ROI) in each of a plurality histology images or electronic images of a cancerous organ, each image annotated with normal and tumor regions indications;
collapsing each ROI in the plurality of ROIs in a transverse direction associated with a transverse width of the ROI to produce a 1 dimensional signal in an analysis direction associated with an analysis width;
convolving the 1 dimensional signal in each ROI with a set of complex sinusoidal functions to compute a simulated MRH spectrum as a function of wavelength;
computing simulated spectra from each ROI in the plurality of ROIs over a plurality of k-values producing a plurality of datasets corresponding to each image in the plurality of images;
selecting one or more neighborhoods of each ROI from the set of 4 nearest neighbors, 8 nearest neighbors 24 nearest neighbors or 16 non-adjacent neighbors;
receiving the simulated spectra from each ROI in a neural network to produce a two-element probability vector that the input spectrum for that ROI is from normal tissue and the probability that it is from tumor, with these values summing to 1, training the network by comparison of the resulting probability vector with the annotated with normal and tumor regions indications for that ROI;
obtaining MRH data producing actual spectra on a plurality of VOIs in a 3D organ;
collapsing the VOIs along two dimensions, leaving a 1-D signal from which a spectrum at wavelengths matching the k-values of the simulated spectra and
receiving the 1-D signal from the actual spectra from each VOI and a selected neighborhood of that VOI in the neural network to produce a two-element probability vector that the spectrum for that VOI is from normal tissue and the probability that it is from tumor, with these values summing to 1.

10. A method for magnetic resonance histopathology (MRH) measurements by using annotated histology slides to train neural network classifiers, the method comprising:
creating a Pseudo whole-organ digitized H&E-stained histology slide, with annotations indicating tumor, Prostatic intraepithelial neoplasia (PIN), and atypical glands;
converting the tumor annotations to an image mask used to label regions of interest (ROI) as normal or tumor;
segmenting the whole-organ slide into a peripheral zone;
dividing the peripheral zone into a plurality of ROIs;
identifying an example normal ROI and an ROI located within a tumor;
collapsing each ROI in the plurality of ROIs in one dimension to produce a 1-D position signal;
Fourier transforming the 1D position signals of the plurality of ROI to produce a simulated MRH spectrum; and,
comparing the spectra from the normal and tumor ROIs, demonstrating notable differences in texture;
providing the simulated MRH spectrum to a neural network classifier with intensities at a selected plurality of wavelengths;
outputting a vector from the neural network classifier of probabilities that the corresponding sample is normal or tumor;
obtaining MRH data producing actual MRH spectra on a plurality of VOIs in a 3D organ; and
receiving the actual spectra from each VOI in the neural network to produce a two-element probability vector that the spectrum for that VOI is from normal tissue and the probability that it is from tumor, with these values summing to 1.

11. The method of claim 10 further wherein
the selected plurality of wavelengths are evenly sampled across the simulated MRH spectrum, enabling differentiation between normal and tumor tissue;
using as input to the classifier, intensities at the selected plurality of wavelengths, extracted from the simulated MRH spectrum.

12. The method of claim 10 further comprising:
employing neighboring-ROI inputs to incorporate an effect of spatial information in the step of collapsing each ROI wherein the neighboring-ROI inputs are selected from one of the following; rod shaped inputs comprising the ROI with one nearest neighbor on either side, giving a (3,1) input size; the ROI with two neighbors on either side, giving a (5,1) input size; the ROI with three neighbors on either side, giving a (7,1) input size; or square-shaped inputs, one of the ROI with one layer of neighbors in all directions, forming a (3,3) sized input; the ROI with two layers of neighbors, forming a (5,5) input size; the ROI with three layers of neighbors, forming a (7,7) input size.
